SEQUENCE LISTING (15 sequences)

SEQ ID NO: 1 - DNA, 24 bp, Artificial Sequence
  Description of Artificial Sequence Synthetic primer
ttgctacttgtgattgctccatgt

SEQ ID NO: 2 - DNA, 26 bp, Artificial Sequence
  Description of Artificial Sequence Synthetic primer
cttgccacacaatcatcacctgccat

SEQ ID NO: 3 - DNA, 32 bp, Artificial Sequence
  Description of Artificial Sequence Synthetic primer
actacttgaagcactcaaggcaagctttattg

SEQ ID NO: 4 - DNA, 25 bp, Artificial Sequence
  Description of Artificial Sequence Synthetic primer
tagagccctggaagcatccaggaag

SEQ ID NO: 5 - DNA, 27 bp, Artificial Sequence
  Description of Artificial Sequence Synthetic primer
ttaggcatctcctatggcaggaagaag

SEQ ID NO: 6 - DNA, 23 bp, Artificial Sequence
  Description of Artificial Sequence Synthetic primer
gtctcgagatactgctcccaccc

SEQ ID NO: 7 - DNA, 34 bp, Artificial Sequence
  Description of Artificial Sequence Synthetic primer
ccttgagtgcttcaagtagtgtgtgcccgtctgt

SEQ ID NO: 8 - DNA, 29 bp, Artificial Sequence
  Description of Artificial Sequence Synthetic primer
gtagtgtgtgcccgtctgttgtgtgactc

SEQ ID NO: 9 - DNA, 29 bp, Artificial Sequence
  Description of Artificial Sequence Synthetic primer
caatcatcacctgccatctgttttccata

SEQ ID NO: 10 - DNA, 23 bp, Artificial Sequence
  Description of Artificial Sequence Synthetic primer
acagcagtacaaatggcagtatt

SEQ ID NO: 11 - DNA, 28 bp, Artificial Sequence
  Description of Artificial Sequence Synthetic primer
tggaaaggtgaaggggcagtagtaatac

SEQ ID NO: 12 - DNA, 30 bp, Artificial Sequence
  Description of Artificial Sequence Synthetic primer
tgaagcactcaaggcaagctttattgaggc

SEQ ID NO: 13 - DNA, 9692 bp, Artificial Sequence
  Description of Artificial Sequence Synthetic polynucleotide
tggaagggatgatttactcccagaaaagacaagatatccttgatctgtggatgtatcacacacaaggttacttccctgattggcagaactacacaccagggccaggggtcagatatccactgacctttggatggtgcttcaagctagtaccagttgagcaagagaagataaaagccaatgaaaatgagaacagcaagttgttacaccccataagcctgcatgggatggatgacccagaaggagaagtgttacagtggaagtttgacagccgcctagcatttcatcacgtggcccgagagctgcatccggagtattacaaggactgctgacaccgagctttctacaagggactttccgctggggactttccagggaaggcgtgacctgggcggtactggggagtggcgagccctcagatgctgcatataagcagctgcttttgcctgtactgggtctctctggctagaccagatctgagcctgggagctctctggctagctagggaacccactgcttaagcctcaataaagcttgtcttgagtgcttcaagtagtgtgtgcccgtctgttgtgtgactctggtaactagagatccctcagaccattgtagtcagtgtggaaaatctctagcagtggcgcccgaacagggacttgaaagcgaaagagaaaccagaggagatctctcgacgcaggactcggcttgctgaagcgcgcacggcaagaggcgaggggcggcgactggtgagtacgccaaatttttgactagcggaggctagaaggagagagatgggtgcgagagcgtcagtattaagcgggggagaattagatagatgggaaaaaattcggttaaggccagggggaaagaaacaatataaattaaaacatatagtatgggcaagcagggagttagaacgattcgcacttaatcctggcctattagaaacatcagaaggatgtagacaaatattgggacagctacaaccagccattcagacaggatcagaagaattaagatcattatttaatacagtagcaaccctctattgtgtgcatcaaaggatagacataaaagacaccaaggaagctttagataagatagaggaagagcaaaacaaaagtaagaaaaaggtacagcaagaagcagctgacacaggaagcaaaagtcaggtcagccaaaattaccccatagtgcagaacatccaggggcaaatggtacaccagcccatatcacctagaactttaaatgcatgggtaaaagtagtagaagagaaggctttcagcccagaagtaatacccatgttttcagcattagcagaaggagccaccccacaggatttaaacaccatgctaaacacagtggggggacatcaagcagctatgcaaatgttaaaagagacaatcaatgaggaagctgcagaatgggatagagtacacccagtgcatgcagggcctgttgcaccaggccagatgagagaaccaaggggaagtgacatagcaggaactactagcacccttcaagaacaaataggatggatgacaaataatccacctatcccagtaggagagatctataaaagatggataatcctgggattaaataaaatagtaaggatgtatagtcctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaatccagattgtaaaaccattctaaaagcattgggaccagcagctacactagaagaaatgatgacagcatgtcagggagtgggaggacccggccataaagcaagagttttggctgaagcaatgagccaaatgaattcagctaccataatgatgcagaaaagtaatttcaggaaccaaagaaagattgttaagtgtttcaattgtggcaaagaagggcacatagccagaaattgcagggcccccaggaaaaagggctgttggaaatgtggaaaggaaggacaccaaatgaaagattgtactgagagacaggctaattttttagggaaaatctggccttcccacaaggggaggccaggaaactttcttcagagcagaccagagccaacagccccaccagaggagagcttcaggtttggggaagagacaacaactccctctcagaagcaggaaccgatagacaaggaactgtatcctttagcttccctcagatcactctttggcaacgacccctcgtcacaataaaaataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcctattagtcctattgaaactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccaatattcgccataaagaagaaagacagtactagatggagaaaattagtagacttcagagaacttaataagaaaactcaagacttctgggaaattcaattaggaataccacatcccgcagggttaaaaaagaaaaaatcagtgacagtactggatgtgggtgatgcatatttttcagttcccttagataaagacttcaggaagtatactgcatttaccatacctagtataaacaatgagacaccagggattaggtatcagtacaatgtgctcccacagggatggaaaggatcaccagcaatatttcaaagtagcatgacaaaaatcttagatccttttagaaaacaaaatccagacatagttatctatcaatacatggatgatttgtatgtaggatctgacttagaaataggacagcatagagccaagatagaggaactgagacaacatctgttgaggtggggattcactacaccggacaaaaaacatcagaaagaacctccattcctttggatgggttatgaactccatcctgataaatggacagtacagcctatagtgctgccagaaaaagacagctggactgtcaatgagatacagaagttagtgggaaaattgaattgggcaagtcagatttacccagggattaaagtaaggcacttatgtaaactccttaggggaaccaaagcactaacagaagtagtaccactaacagcagaagcagagctagaattggcagaaaacagggagattctaagagaaccagtacatggagtgtattatgacccatcaaaagacttaatagcagaattacagaagcaggggcaaggccaatggacatatcaaatttatcaagagccatttaaaaatctgaaaacaggaaaatatgcaagaatgaagggtgcccacactaatgatgtaaaacaattaacagaggcagtgcaaaaaataacccaagaaagcatagtaatatggggaaagactcctaaattcaaactacccatacaaaaagaaacatgggaaacatggtggacagagtattggcaagccacctggattcctgagtgggagtttgtcaatacccctcccttagtgaaattatggtaccagttagagaaagaacccatagtaggagcagaaactttctatgtagatggggcagctaacagggaaactaaattaggaaaagcaggatatgttactgatagaggaagacaaaaggttgtctccctaactgacacaacaaatcagaagactgagttacaagcaattcatctagctttgcaggattcgggattagaagtaaacatagtaacagactcacaatatgcattaggaatcattcaagcacaaccagataaaagtgaatcagagttagtcagtcaaataatagaacagttaataaagaaggaaaagatctacttggcatgggtaccggcacacaaaggaattggaggaaatgaaagggtagataaattagtcagtgctggaatcagaaaagtactatttttagatggaatagaaaaagcccaagatgaacatgagaaatatcacagtaattggagagcaatggctagtgattttaacctgccacctatagtagcaaaagaaatagtagccagctgtgataaatgtcagctaaaaggagaagccatacatggacaagtagactgtagtccagaaatatggcaactagattgtacacatttagaaggaaaaataatcatagtagcagtgcatgtagccagtggatatatagaagcagaagttattccagcagagacagggcaggaaacagcatactttatcttaaaattagcaggaagatggccagtaaaaacaatacatacagacaatggccccaatttcaccagtactgcagttaaggccgcctgttggtgggcggggatcaagcaggaatttggcattccctacaatccccaaagtcaaggagtagtagaatccatgaataaagaattaaagaaaattatagaacaagtaagagatcaggctgaacttcttaaaacagcagtacaaatggcagttttcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatcccctttggaaaggaccagcaaagctgctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggcacagtctagtaaaataccatatgcatatttccaagaaagctaagggatggtcttatagacatcattatgaaagcactcatccaagaataagttcagaagtatacatcccactaggggatgctaaattggtaataacaacatattggggtctgcatccaggagaaagagactggcagttgggtcatggagtctccatagaatggaggaaagagagatatagcacacaagtagatcctaacctagcagaccgcctaattcattggtattactttgattgtttttcagactctgctataagaaaggccatattaggacgtatagttagtcctaggtgtgaatatcaagcaggacataacaaggtaggatctctacagtacttggcactaacagcattaataaaaccaaaaagaacaaagacacctttgcctagtgttgcaaaactgacagaggacagatggaacaagccccagaagaccaagggccacaaagggagccatacactgactgggcactagagcttttagaggaacttaagaatgaagctatgagacatttccctaggatatggctccatggcttggggcaacatatctatgaaacttatggggatacttgggcaggagtggaagccataataagaattctgcaacaactgctgtttattcatttcagaattggatgtcgacatagcagaataggcattattcaacagaggagagcaagaaatggagccagtagatcctagactagagccctggaagcatccaggaagccagcctaagactgcttgtaactcttgctattgtaaaaagtgttgcttacattgccaagtttgtttcatgaaaaaaggcttaggcatctcctatggcaggaagaagcggagacagcgacgaagatctcctccagacagtcagactaatcaagcttctctatcaaagcagtaagtagtacatgtaatgtcatctttagatatattaacaatagtagcaatagtagcattagtaatagtaggaataatagcaatagttgtgtgggccatagtattcatagaatataggagaatattaagacaaagaaaaataaacaggttaattgatagaataagagaaagagcagaagacagtggcaatgagagtgagggggatcaggaagaattattggcactgggggagatggggcatctggagatggggcatgatgctccttgggatgttaatgatctataatgctacaggacaattgtgggtcacagtctattatggggtacctgtgtggaaagaagcaaccaccactttattttgtgcatcagatgctaaagcatatgatacagaggtacataatgtttgggccacacatgcctgtgtacccacagaccccaacccacaagaagtagaattggtaaatgtgacagaaaattttaacatgtgggacaataaaatggtagaacagatgcatgaagacatagtcagtttatgggatcagagcctaaagccatgtgtaaaattaaccccactctgtgttactttaaattgcactgatttgaggaatgctactaatacacataataatgttactaatggaggggaaatgaaaaactgctctttcaatgtcaccacagaggtaagagataaggtgaagaaagaatatgcacttttttacacacttgatatagtacaaatagataatactagctataggatgacaagttgtaacacctcagtcattacacaggcctgtccaaaggtaacctttgagccaattcccatacattattgtgccccggctggttttgcgattctaaagtgtaaagataagaagttcaagggaacaggatcatgtacaaatgtcagcacagtacaatgtacacatggaattaagccagtagtatcaactcaactgctgttaaatggcagtctagcagaagaagaggtagtaattagatctgagaatttcacagacaatactaaaaacataatagtacagctgaatgaagctgtagaaattaattgtacaagactcaacaacaacacaagaaaaagtatacatataggaccagggagagcattttatacaacaggagacataataggagatataagacaagcacattgtaaccttagtagggcagcatggaatacaactttacaaaagatagctgaaaaattaacagaacaatatggaaataaaacaataagctttaatcgatcctcaggaggggacctagaagttgtaatgcataattttaattgtggaggggaatttttctactgtaatacaacagaactgtttaatagtacttggaatggtactgaagtggtaaatatcaatgaaggaaatgacaatatcactcttccatgcagaataaaacaaattataaacatgtggcaggaagtaggaaaagcaatgtatgcccctcccatcagaggaccaattaaatgttcatcgaatattacagggctaatattaacaagagatggtggtactaatgacaccaccgaggtcttcagacctgcaggaggagatatgatggacaattggagaagtgaactatataaatataaagtggtgaaaattgaaccattaggagtagcacccaccaaggcaaagagaagagtggtgcagagagaaaaaagagcaataacaataggagcggtgttccttgggttcttgggaacagcaggaagcactatgggcgcagcgtccgtggcgctgacggtacaggccagacaattattgtctggtatagtgcaacagcaaagcaacttgttgagagctattgaggcgcaacagcatatgttgcaactcacagtctggggcatcaagcagctccaggcaagagtcctggctgtggaaagatacctaaaggatcaacagctcctggggatttggggttgctctggaaaactcatttgcaccactgctgtgccttggaatactagttggagtaataaaaccctggaagagatttggaataacatgacctggatgcagtgggacagagaaattaacaattacacaaacttaatatacaacttaattgaacaatcgcaaaaccagcaagaaaagaatgaacaagaattattggaattggataaatgggcaagtctgtggagttggtttgacataacaaattggctgtggtatataaaaatattcataatgatagtaggaggcttagtaggtttaagaatagtttttactgtactttctatagtgaatagagttaggcagggatattcaccattatcgtttcagacccgctgcccagccccgaggggacccgacaggcccgaaggaatagaagaagaaggtggagagagaggcagagacagatccggtcgcttagtggatggcttattagcactcatctgggacgatctacggagcctgtgcctcttcagctaccaccgcttgagagacttactcttgattacaacgaggattgtggaacttctgggacgcagggggtgggagaccctcaaatattggtggaatctcctgcaatattggagtcaggaactaaggaatagtgctgttagcttgcttaattttacagccgtagcagtagcagagggaacagataggattatagaatttgtacaaagagttggtagagcttttcttcacatacctagaagaataagacagggcttcgaaagggctttgctataagatgggtggcaagtggtcaaaaagcagcgtggttggatggcctagaataagggaaagaatgaaacgagctgaaccagcagcagatggggtgggagcagcatctcgagacctggaaaaacatggagcaatcacaagtagcaatacagcagctaccaatgctgattgtgcctgggtagaagcacaagaagatggggaggaggtgggttttccagtcagacctcaggtacctttaaggccaatgacatataagggagctttagatcttagccactttttaaaagaaaaggggggactggaagggatgatttactcccagaaaagacaagatatccttgatctgtggatgtatcacacacaaggttacttccctgattggcagaactacacaccagggccaggggtcagatatccactgacctttggatggtgcttcaagctagtaccagttgagcaagagaagataaaagccaatgaaaatgagaacagcaagttgttacaccccataagcctgcatgggatggatgacccagaaggagaagtgttacagtggaagtttgacagccgcctagcatttcatcacgtggcccgagagctgcatccggagtattacaaggactgctgacaccgagctttctacaagggactttccgctggggactttccagggaaggcgtgacctgggcggtactggggagtggcgagccctcagatgctgcatataagcagctgcttttgcctgtactgggtctctctggctagaccagatctgagcctgggagctctctggctagctagggaacccactgcttaagcctcaataaagcttgtcttgagtgcttcaagtagtgtgtgcccgtctgttgtgtgactctggtaactagagatccctcagaccattgtagtcagtgtggaaaatctctagc

SEQ ID NO: 14 - DNA, 2544 bp, Human immunodeficiency virus 1
atgagagtgagggggatcaggaagaattattggcactgggggagatggggcatctggagatggggcatgatgctccttgggatgttaatgatctataatgctacaggacaattgtgggtcacagtctattatggggtacctgtgtggaaagaagcaaccaccactttattttgtgcatcagatgctaaagcatatgatacagaggtacataatgtttgggccacacatgcctgtgtacccacagaccccaacccacaagaagtagaattggtaaatgtgacagaaaattttaacatgtgggacaataaaatggtagaacagatgcatgaagacatagtcagtttatgggatcagagcctaaagccatgtgtaaaattaaccccactctgtgttactttaaattgcactgatttgaggaatgctactaatacacataataatgttactaatggaggggaaatgaaaaactgctctttcaatgtcaccacagaggtaagagataaggtgaagaaagaatatgcacttttttacacacttgatatagtacaaatagataatactagctataggatgacaagttgtaacacctcagtcattacacaggcctgtccaaaggtaacctttgagccaattcccatacattattgtgccccggctggttttgcgattctaaagtgtaaagataagaagttcaagggaacaggatcatgtacaaatgtcagcacagtacaatgtacacatggaattaagccagtagtatcaactcaactgctgttaaatggcagtctagcagaagaagaggtagtaattagatctgagaatttcacagacaatactaaaaacataatagtacagctgaatgaagctgtagaaattaattgtacaagactcaacaacaacacaagaaaaagtatacatataggaccagggagagcattttatacaacaggagacataataggagatataagacaagcacattgtaaccttagtagggcagcatggaatacaactttacaaaagatagctgaaaaattaacagaacaatatggaaataaaacaataagctttaatcgatcctcaggaggggacctagaagttgtaatgcataattttaattgtggaggggaatttttctactgtaatacaacagaactgtttaatagtacttggaatggtactgaagtggtaaatatcaatgaaggaaatgacaatatcactcttccatgcagaataaaacaaattataaacatgtggcaggaagtaggaaaagcaatgtatgcccctcccatcagaggaccaattaaatgttcatcgaatattacagggctaatattaacaagagatggtggtactaatgacaccaccgaggtcttcagacctgcaggaggagatatgatggacaattggagaagtgaactatataaatataaagtggtgaaaattgaaccattaggagtagcacccaccaaggcaaagagaagagtggtgcagagagaaaaaagagcaataacaataggagcggtgttccttgggttcttgggaacagcaggaagcactatgggcgcagcgtccgtggcgctgacggtacaggccagacaattattgtctggtatagtgcaacagcaaagcaacttgttgagagctattgaggcgcaacagcatatgttgcaactcacagtctggggcatcaagcagctccaggcaagagtcctggctgtggaaagatacctaaaggatcaacagctcctggggatttggggttgctctggaaaactcatttgcaccactgctgtgccttggaatactagttggagtaataaaaccctggaagagatttggaataacatgacctggatgcagtgggacagagaaattaacaattacacaaacttaatatacaacttaattgaacaatcgcaaaaccagcaagaaaagaatgaacaagaattattggaattggataaatgggcaagtctgtggagttggtttgacataacaaattggctgtggtatataaaaatattcataatgatagtaggaggcttagtaggtttaagaatagtttttactgtactttctatagtgaatagagttaggcagggatattcaccattatcgtttcagacccgctgcccagccccgaggggacccgacaggcccgaaggaatagaagaagaaggtggagagagaggcagagacagatccggtcgcttagtggatggcttattagcactcatctgggacgatctacggagcctgtgcctcttcagctaccaccgcttgagagacttactcttgattacaacgaggattgtggaacttctgggacgcagggggtgggagaccctcaaatattggtggaatctcctgcaatattggagtcaggaactaaggaatagtgctgttagcttgcttaattttacagccgtagcagtagcagagggaacagataggattatagaatttgtacaaagagttggtagagcttttcttcacatacctagaagaataagacagggcttcgaaagggctttgctataa

SEQ ID NO: 15 - protein, 847 aa, Human immunodeficiency virus 1
MRVRGIRKNYWHWGRWGIWRWGMMLLGMLMIYNATGQLWVTVYYGVPVWKEATTTLFCASDAKAYDTEVHNVWATHACVPTDPNPQEVELVNVTENFNMWDNKMVEQMHEDIVSLWDQSLKPCVKLTPLCVTLNCTDLRNATNTHNNVTNGGEMKNCSFNVTTEVRDKVKKEYALFYTLDIVQIDNTSYRMTSCNTSVITQACPKVTFEPIPIHYCAPAGFAILKCKDKKFKGTGSCTNVSTVQCTHGIKPVVSTQLLLNGSLAEEEVVIRSENFTDNTKNIIVQLNEAVEINCTRLNNNTRKSIHIGPGRAFYTTGDIIGDIRQAHCNLSRAAWNTTLQKIAEKLTEQYGNKTISFNRSSGGDLEVVMHNFNCGGEFFYCNTTELFNSTWNGTEVVNINEGNDNITLPCRIKQIINMWQEVGKAMYAPPIRGPIKCSSNITGLILTRDGGTNDTTEVFRPAGGDMMDNWRSELYKYKVVKIEPLGVAPTKAKRRVVQREKRAITIGAVFLGFLGTAGSTMGAASVALTVQARQLLSGIVQQQSNLLRAIEAQQHMLQLTVWGIKQLQARVLAVERYLKDQQLLGIWGCSGKLICTTAVPWNTSWSNKTLEEIWNNMTWMQWDREINNYTNLIYNLIEQSQNQQEKNEQELLELDKWASLWSWFDITNWLWYIKIFIMIVGGLVGLRIVFTVLSIVNRVRQGYSPLSFQTRCPAPRGPDRPEGIEEEGGERGRDRSGRLVDGLLALIWDDLRSLCLFSYHRLRDLLLITTRIVELLGRRGWETLKYWWNLLQYWSQELRNSAVSLLNFTAVAVAEGTDRIIEFVQRVGRAFLHIPRRIRQGFERALL

(19) United States
(12) Patent Application Publication
(10) Pub. No.: US 2013/0177583 A1
(43) Pub. Date: Jul. 11, 2013

(54) MOLECULAR CLONE OF HIV-1

(71) Applicants: George M. Shaw, Birmingham, AL (US); Beatrice H. Hahn, Birmingham, AL (US); Hui Li, Birmingham, AL (US); Barton F. Haynes, Durham, NC (US); Martin Markowitz, New York, NY (US)

(72) Inventors: George M. Shaw, Birmingham, AL (US); Beatrice H. Hahn, Birmingham, AL (US); Hui Li, Birmingham, AL (US); Barton F. Haynes, Durham, NC (US); Martin Markowitz, New York, NY (US)

(73) Assignees: Duke University, Durham, NC (US); The UAB Research Foundation, Birmingham, AL (US)

(21) Appl. No.: 13/582,319

(22) PCT Filed: Mar. 3, 2011
(86) PCT No.: PCT/US2011/000411

§ 371 Date: Mar. 28, 2013

Related U.S. Application Data
(60) Provisional application No. 61/282,581, filed on Mar. 3, 2010.

(51) Int. Cl. A61K 39/21

(52) U.S. Cl. CPC A61K 39/21
USPC 424/188.1; 530/350; 536/23.72; 435/320.1; 530/387.9; 435/235.1

(57) ABSTRACT

The present invention relates, in general, to HIV-1 and, in particular, to a molecular clone of HIV-1. The invention further relates to methods of inducing an immune response to HIV-1 in a patient and to immunogens suitable for use in such methods. The invention also relates to anti-HIV-1 antibodies and to methods of using same to prevent or treat HIV-infection.

This application claims priority from U.S. Provisional Application No. 61/282,581, filed Mar. 3, 2010, the entire content of which is incorporated herein by reference.

This invention was made with government support under Grant Nos. AI 67854 and AI 041534 awarded by the National Institutes of Health. The government has certain rights in the invention.

### TECHNICAL FIELD

The present invention relates, in general, to HIV-1 and, in particular, to a molecular clone of HIV-1. The invention further relates to methods of inducing an immune response to HIV-1 in a patient and to immunogens suitable for use in such methods. The invention also relates to anti-HIV-1 antibodies and to methods of using same to prevent or treat HIV-infection.

### BACKGROUND

An effective sterilizing HIV-1 vaccine ideally should target virus in the earliest stages of transmission, prior to dissemination and establishment of persistent infection (Haase, Nat. Rev. Immunol. 5:783-792 (2005), Hladik et al, Nat. Rev. Immunol. 8:447-457 (2008), Pope et al, Nat. Med. 9:847-852 (2003), Shattock et al, Nat. Rev. Microbiol. 1:25-34 (2003)). To be broadly protective, such a vaccine must defend against a genetically diverse set of viruses transmitted by different sexual practices and risk behaviors. Results from the recently reported ‘Thai Trial’ RV144 of an experimental HIV-1 vaccine showed a decrease in virus acquisition of 31.2% (p=0.04) based on a modified intention-to-treat analysis and a trend for greater vaccine effectiveness in those subjects identified as practicing lower risk behaviors (Rerks-Ngarm et al, N. Engl. J. Med. 361:2209-2220 (2009)). These findings suggest that an HIV-1 vaccine might be more efficacious in preventing infection by some exposure routes than others (Rerks-Ngarm et al, N. Engl. J. Med. 361:2209-2220 (2009), Dolin, N. Engl. J. Med. 361:2279-2280 (2009), Letvin, Science 326:1196-98 (2009)).

Recently, single genome amplification (SGA), direct sequencing, and a model of random virus evolution were employed to identify those viruses responsible for transmission and productive clinical infection in several largely heterosexual cohorts with acute HIV-1 subtype A, B or C infection (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008), Salazar-Gonzalez et al, J. Virol. 82:3952-3970 (2008), Salazar-Gonzalez et al, J. Exp. Med. 205:1273-1289 (2009)) and in Indian rhesus macaques inoculated intra-rectally with SIVmac251 or SIVsmmE660 (Keele et al, J. Exp. Med. 206:1117-1134 (2009)). This experimental approach allows for the distinction of transmitted/founder viruses that differ by as little as a single nucleotide (Keele et al, Proc. Natl. Acad. Sci. USA 105:7552-7557 (2008), Keele et al, J. Exp. Med. 206:1117-1134 (2009)). SGA-direct sequencing also makes possible the identification of transmitted viral sequences in linked transmissions, thereby enabling the unambiguous tracking of viruses from donor to recipient across mucosal surfaces (Haaland et al, PLoS Pathog. 5:e1000274 (2009), Keele et al, J. Exp. Med. 206:1117-1134 (2009)), and the molecular cloning and analysis of those viruses actually responsible for productive clinical infection (Salazar-Gonzalez et al, J. Exp. Med. 205:1273-1289 (2009)).

Previous studies based on different experimental approaches have been informative with respect to determining the overall extent of viral diversity present in acute and early infection as a surrogate for identifying and quantifying transmitted viruses (Derdeyn et al, Science 303:2019-2022 (2004), Gottlieb et al, J. Infect. Dis. 197:1011-1015 (2008), Grobler et al, J. Infect. Dis. 190:1355-1359 (2004), Learn et al, J. Virol. 76:11953-11959 (2002), Long et al, Nat. Med. 6:71-75 (2000), Poss et al, J. Virol. 69:8118-8122 (1995), Ritola et al, J. Virol. 78:11208-11218 (2004), Sagar et al, J. Virol. 78:7279-7283 (2004), Wolfs et al, Virology 189:103-110 (1992), Wolinsky et al, Science, 255:1134-1137 (1992), Zhu et al, Science 261:1179-1181 (1993)). Such studies generally described new infections as being either “homogeneous,” presumably reflecting infection by one or few viruses, or “heterogeneous,” suggesting infection by more viruses. Based on these studies, a substantial bottleneck in virus transmission was recognized to exist, since the genetic complexity of viral quasispecies in the blood of chronically infected individuals was generally much greater than that in acutely infected subjects. Evidence for a bottleneck in virus transmission, although not necessarily at the mucosal interface, was further suggested by the longstanding observation that most new infections are caused by R5 tropic viruses and not by X4 tropic viruses, which are common in chronic infection (Margolis et al, Nat. Rev. Microbiol. 4:312-317 (2006), Moore et al, AIDS Res. Hum. Retroviruses 20:111-126 (2004), Richman et al, J. Infect. Dis. 169:968-974 (1994)). These studies and others in the related Indian rhesus macaque-SIV infection model (Keele et al, J. Exp. Med. 206:1117-1134 (2009), Li et al, Nature 434:1148-1152 (2005), Li et al, Nature 458:1034-1038 (2009), Li et al, Science 323:1726-1729 (2009), Miller et al, J. Virol. 79:9217-9227 (2005)) thus focused attention on the mucosa and submucosa as a potentially important barrier to HIV-1 transmission and a site where critical early virus-host cell interactions leading to transmission and productive clinical infection likely take place (Haase, Nat. Rev. Immunol. 5:783-792 (2005), Hladik et al, Nat. Rev. Immunol. 8:447-457 (2008), Pope et al, Nat. Med. 9:847-852 (2003), Shattock et al, Nat. Rev. Microbiol. 1:25-34 (2003), Margolis et al, Nat. Rev. Microbiol. 4:312-317 (2006), Moore et al, AIDS Res. Hum. Retroviruses 20:111-126 (2004), Hladik et al, Immunity 26:257-270 (2007)). However, it was not until the application of SGA, direct amplicon sequencing, and a model of random virus evolution to the analysis of viral genomes in the acute infection period that actual transmitted/founder viruses could be identified and their numbers precisely estimated (Keele et al, Proc. Natl. Acad. Sci. USA 105:7552-7557 (2008), Keele et al, J. Exp. Med. 206:1117-1134 (2009)). The present invention results, at least in part, from the application of this strategy to a systematic analysis and comparison of multiplicity of HIV-1 infection in men who have sex with men (MSM) versus heterosexuals (HSX).

### SUMMARY OF THE INVENTION

In general, the present invention relates to HIV-1. More specifically, the invention relates to a molecular clone of HIV-1. The invention further relates to methods of inducing an immune response to HIV-1 in a patient and to immunogens suitable for use in such methods. The invention also relates to anti-HIV-1 antibodies and to methods of using same to prevent or treat HIV-infection.

Objects and advantages of the present invention will be clear from the description that follows.

### BRIEF DESCRIPTION OF THE DRAWINGS

FIG. 1. Neighbor-joining (NJ) tree of full-length HIV-1 gp160 env sequences from 28 acutely infected subjects and 2 chronically infected sexual partners. Two chronic-to-acute (LACU9000 to HOBRO961 and AD18 to AD17) and two acute-to-acute (AD77 to AD75 and AD83 to 04013240) transmissions were documented, with donor sequences shown in blue and recipient sequences shown in green. Individual sequences with APOBEC G-to-A hypermutation were excluded from the analysis. Bootstrap values (≧70%) are shown for intra-subject clusters, partner pairs, and additional sequences with evidence of epidemiologic linkage. The horizontal scale bar represents 1.0% genetic distance.

FIGS. 2A-2D. NJ trees and Highlighter plots of env diversity. Full-length gp160 env sequences from four subjects are depicted by NJ tree phylogenies and by Highlighter, a sequence visualization tool that allows tracing of common ancestry between sequences based on individual nucleotide polymorphisms (Keele et al, Proc. Natl. Acad. Sci. USA 105:7552-557 (2006)). Sequences from subject 04013440 (FIG. 2A) showed productive infection by a single virus, from subject 04013211 (FIG. 2B) infection by two closely related viruses, from subject 04013383 (FIG. 2C) infection by two distantly related viruses, and from subject 04013448 (FIG. 2D) infection by four viruses with inter-lineage recombinants denoted by orange symbols in the NJ tree. The horizontal scale bar represents genetic distance.

FIG. 3. Time course of HIV-1 exposure, symptom onset, viral kinetics, and initiation of antiretroviral therapy in subject AD17. ARS, acute retroviral syndrome. HAART, highly active antiretroviral therapy. For purposes of mathematical modeling, a plasma virus load of 10 RNA molecules per milliliter was estimated at day 6.

FIGS. 4A and 4B. NJ trees and Highlighter plots of diversity in 5′ (FIG. 4A) and 3′ (FIG. 4B) half genomes in subject AD17. Blue symbols represent sequences from day 14 and green symbols day 17 as depicted in FIG. 3. Solid ovals represent plasma vRNA derived sequences and solid triangles represent peripheral blood mononuclear cell DNA derived sequences.

FIGS. 5A-5C. Molecular cloning and biological analysis of the transmitted/founder virus from subject AD17. (FIG. 5A) Cloning strategy and genome organization of pAD17.1. (FIG. 5B) Replication of pAD17.1 virus in activated primary human CD4+ lymphocytes (left panel) and monocyte-derived macrophages (right panel) from the same normal blood donor. Results were replicated three times in cells from different donors, each time showing efficient replication of pAD17.1 virus in CD4+ T cells but not in macrophages. (FIG. 5C) pAD17.1 virus infection of JC53BL-13 cells assessed by luciferase expression (Salazar-Gonzalez et al, J. Exp. Med. 206:1273-1289 (2009)) in the absence or presence of the CXCR4 inhibitor AMD3100 (1.2 uM) or the CCR5 inhibitor TAK779 (10 uM) or both. Results from four experiments are expressed as infectivity (mean±1 S.D.) relative to control wells lacking coreceptor inhibitor: NL4.3 is X4 tropic, YU2 is R5 tropic, WEAU1.60 is dual R5/X4 tropic, and pAD17.1 is R5 tropic.

FIG. 6. NJ tree and Highlighter plot of env diversity in subject 04013171. Sequences emanating from ten transmitted/founder viruses are color-coded and identified as variants 1-10. Inter-lineage recombinants are depicted in orange. The horizontal scale bar represents genetic distance.

FIGS. 7A-7C. NJ trees and Highlighter plots of diversity in env gp41 (FIG. 7A), env gp160 (FIG. 7B), and 3′ half (FIG. 7C) genomes in subject 7010100068. Seventy-two 3′ half genomes were amplified and sequenced and segments of each are represented in FIGS. 7A, 7B, and 7C. The progeny of transmitted/founder viruses are color-coded and identifiable as discrete ‘rakes’ of identical or nearly identical sequences (variants 1-7) in the env gp41 segments shown in FIG. 7A. The relatedness of sequences emanating from the seven transmitted/founder viruses is progressively obscured in FIGS. 7B and 7C as longer segments are compared due to inter-lineage recombination. The horizontal scale bar represents genetic distance.

FIG. 8. The genome of the pAD17.1 clone.

FIGS. 9A and 9B. FIG. 9A. The nucleotide sequence encoding the pAD17.1 Env protein. FIG. 9B. The amino acid sequence of the pAD17.1 Env protein.

### DETAILED DESCRIPTION OF THE INVENTION

Understanding the biology of sexual transmission of HIV-1 could contribute importantly to the development of effective vaccines, microbiocides or other prevention measures. However, different routes of virus transmission (vaginal, rectal, penile or oral), different directions of transmission (male-to-female, female-to-male, or male-to-male), and the inaccessibility of these tissues at or near the time of virus transmission make this goal elusive. A strategy has been developed that solves a substantial part of this problem: single genome amplification and sequencing of plasma HIV-1 sequences from acutely infected subjects analyzed in the context of a model of random virus evolution so as to infer the exact nucleotide sequence of actual transmitted/founder virus genomes.

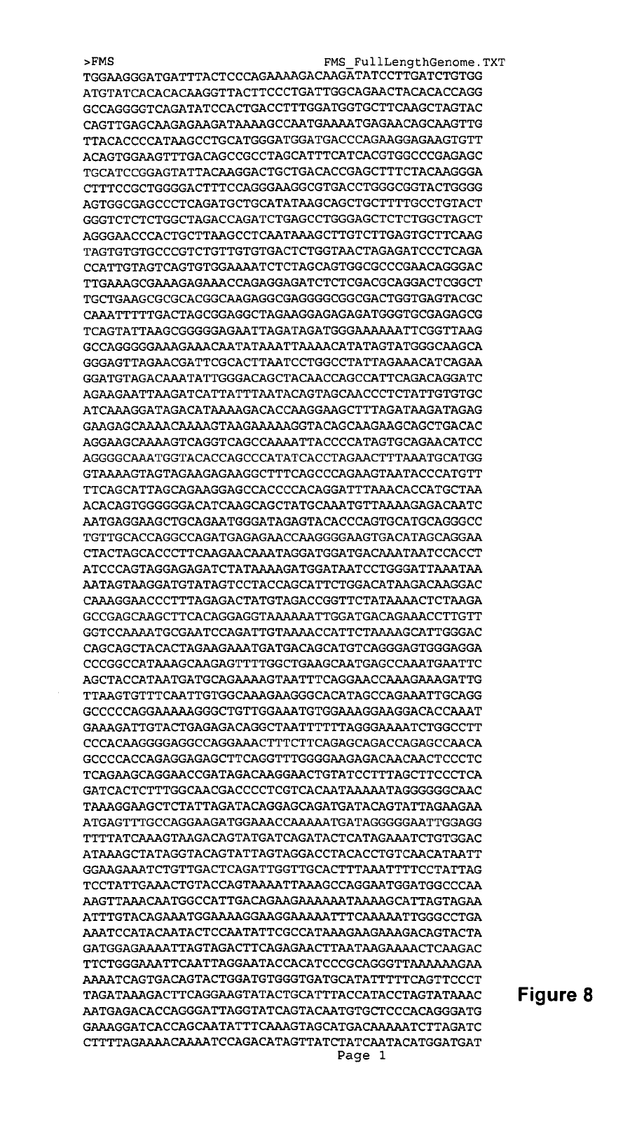

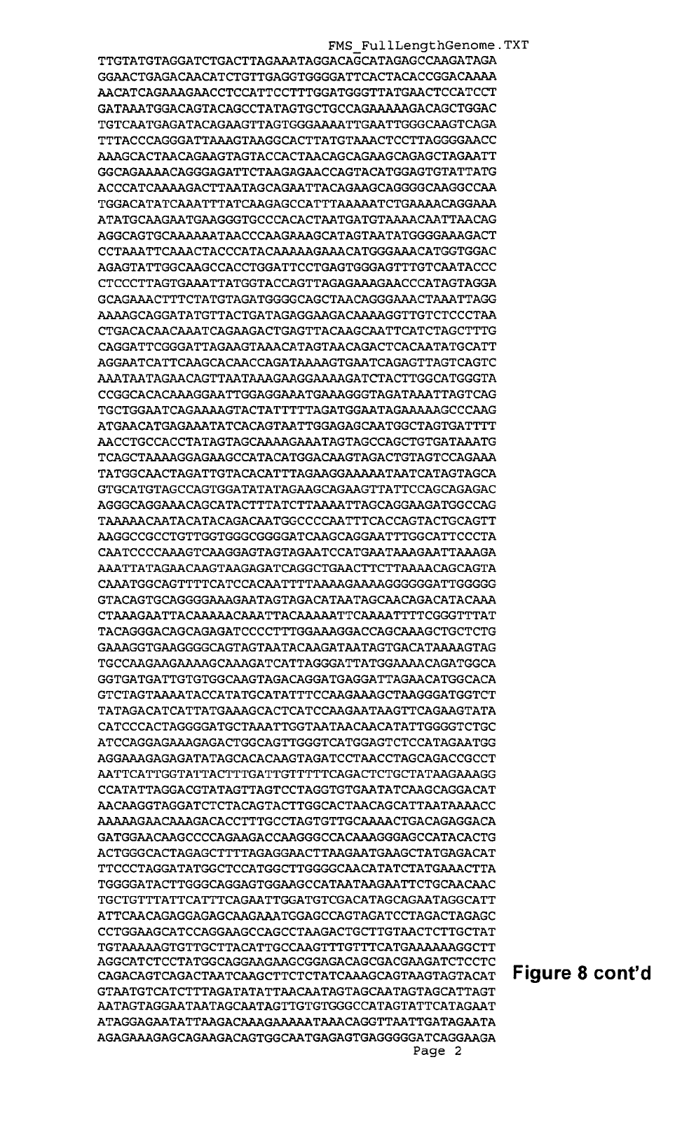

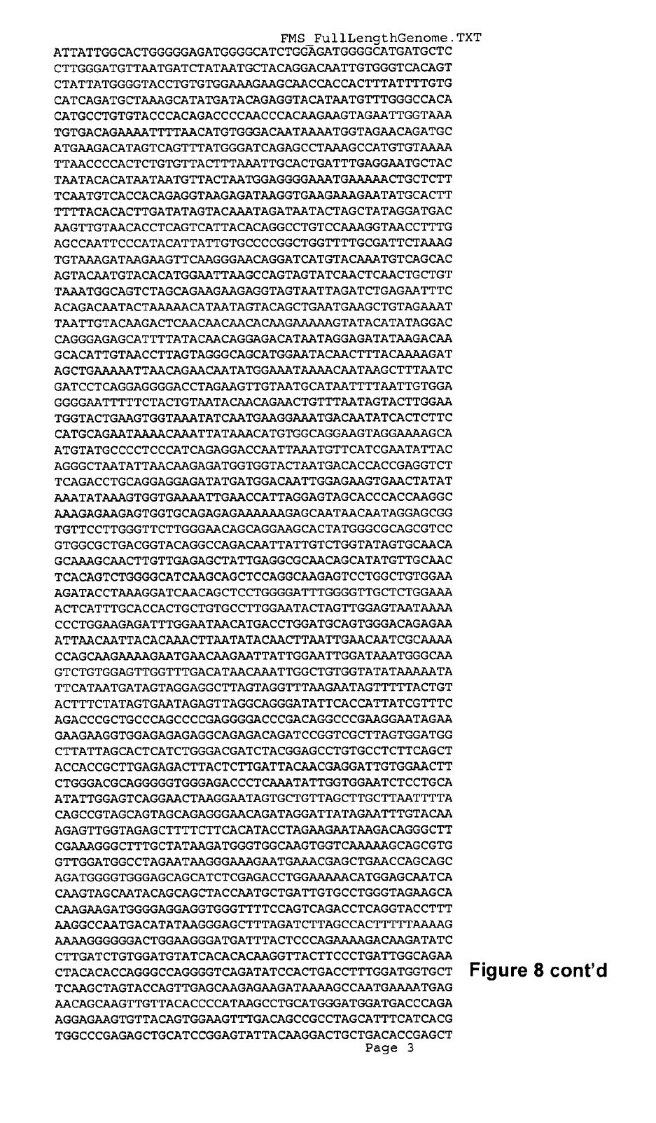

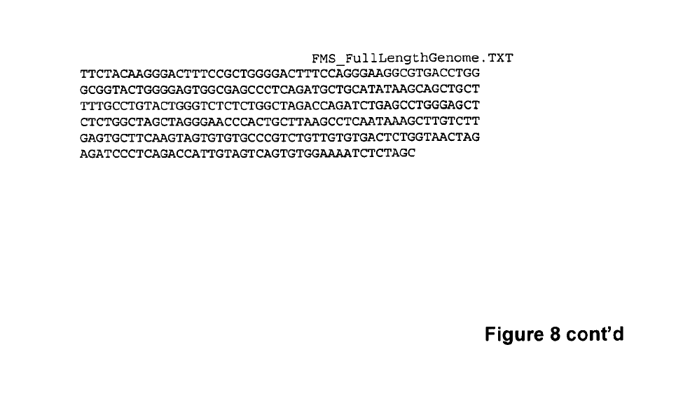

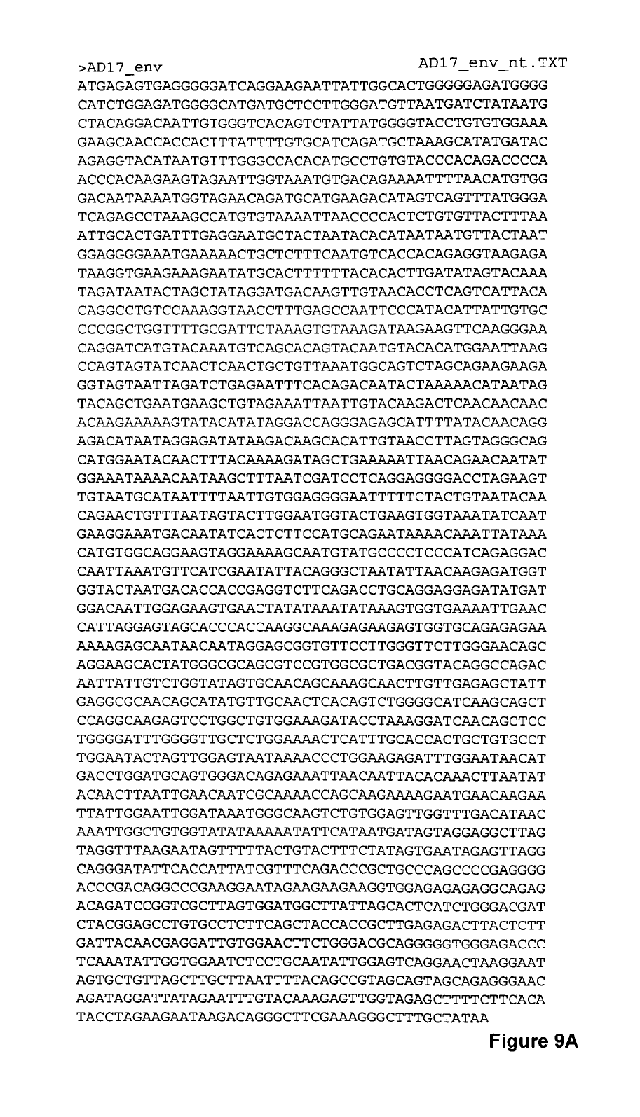

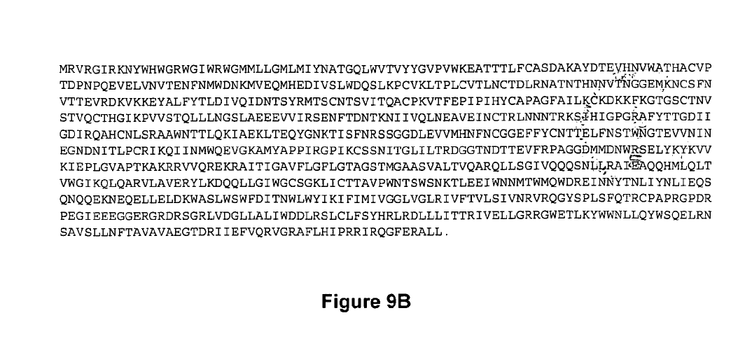

In the study described in the Example that follows, this strategy was applied in a cohort of men who have sex with men (MSM) and it was found that they are twice as likely as heterosexuals to become infected by multiple viruses as opposed to a single virus. Some MSM subjects were infected by as many as 7 to 10 or more genetically distinct viruses as a consequence of a single exposure event. Also described in the Example is the molecular cloning of the first full-length transmitted/founder subtype B HIV-1 virus (pAD17.1) which is replication competent in human cells. The genome of the pAD17.1 clone is set forth in FIG. 8, the nucleotide sequence encoding the Env protein is set forth in FIG. 9A and the amino acid sequence of the Env protein is provided in FIG. 9B. This study provides the first comparative, quantitative analysis of the multiplicity of HIV-1 infection in the two primary risk groups—MSM and heterosexuals—driving the global pandemic.

The present invention relates to HIV Envs from transmitted/founder viruses (e.g., the pAD17.1 virus) and methods of using same as vaccine immunogens. The invention further relates to HIV Envs from transmitted viruses (e.g., the pAD17.1 virus) for use as diagnostic targets in diagnostic tests. The invention further relates to the use of wildtype (WT) transmitted/founder virus sequences (e.g., pAD17.1 sequences) in the preparation of a polyvalent HIV-1 vaccine. Sequences that can be included in such a polyvalent vaccine include WT gag, env, pol, nef and tat sequences.

The immunogens of the invention can be chemically synthesized and purified using methods well known in the art. The immunogens can also be synthesized by well-known recombinant DNA techniques. Nucleic acids encoding the immunogens of the invention can be used as components of, for example, a DNA vaccine wherein the encoding sequence is administered as naked DNA or, for example, a minigene encoding the immunogen can be present in a viral vector. The encoding sequence can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the invention. Expression of the immunogen of the invention can be induced in a patient's own cells, by introduction into those cells of nucleic acids that encode the immunogen, preferably, using codons and promoters that optimize expression in human cells. Examples of methods of making and using DNA vaccines are disclosed in, for example, U.S. Pat. Nos. 5,580,859, 5,589,466, and 5,703,055.

The invention includes compositions comprising an immunologically effective amount of the immunogen of the invention (e.g., the pAD17.1 Env) or fragment thereof (e.g., gp41, gp120, peptides from the membrane proximal region either alone or associated with lipids, or fragments of gp120), or nucleic acid sequence encoding same, in a pharmaceutically acceptable delivery system. The compositions can be used for prevention and/or treatment of immunodeficiency virus infection. The compositions of the invention can be formulated using adjuvants (e.g., alum, AS021 (from GSK), oligo CpGs, MF59 or Emulsigen), emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosa] immunity (e.g., the formulation can be designed for intranasal administration). The present compositions can be administered by any convenient route including subcutaneous, intranasal,. intrarectal, intravaginal, oral, intramuscular, or other parenteral or enteral route, or combinations thereof. The immunogens can be administered in an amount sufficient to induce an immune response, e.g., as a single dose or multiple doses. Optimum immunization schedules can be readily determined by the ordinarily skilled artisan and can vary with the patient, the composition and the effect sought.

Examples of compositions and administration regimens of the invention include consensus or mosaic gag genes and consensus or mosaic nef genes and consensus or mosaic pol genes and consensus Env with wild-type transmitted/founder virus Env (e.g. pAD17.1 Env) or mosaic Env with wild-type transmitted/founder virus Env (e.g., pAD17.1 Env), expressed as, for example, a DNA prime recombinant Vesicular stomatitis virus boost and a recombinant Envelope protein boost for antibody, a poxvirus prime such as NYVAC and a protein AD 17.1 envelope oligomer boost, or fragment thereof, or DNA prime recombinant adenovirus boost and Envelope protein boost, or, for just antibody induction, only the recombinant envelope gp120 or gp140 as a protein in an adjuvant. (See U.S. application Ser. No. 10/572,638 and PCT/US2006/032907.)

The invention contemplates the direct use of both the immunogen of the invention and/or nucleic acid encoding same and/or the immunogen expressed as a minigene in the vectors indicated above. For example, a minigene encoding the immunogen can be used as a prime and/or boost.

It will be appreciated from a reading of this disclosure that the whole Envelope gene can be used or portions thereof (i.e., as minigenes). In the case of expressed proteins, protein subunits can be used.

As pointed out above, the invention also relates to diagnostic targets and diagnostic tests. For example, Envelope (e.g., the pAD17.1 Env) can be expressed by transient or stable transfection of mammalian cells (or they can be expressed, for example, as recombinant Vaccinia virus proteins). The protein can be used in ELISA, Luminex bead test, or other diagnostic tests to detect antibodies to the transmitted/founder virus in a biological sample from a patient at the earliest stage of HIV infection.

The present invention also relates to antibodies specific for transmitted/founder viral sequences (e.g. pAD17.1 sequences), and fragments of such antibodies, and to methods of using same to inhibit infection of cells of a subject by HIV-1. The method comprises administering to the subject (e.g., a human subject) the HIV-1 specific antibody, or fragment thereof, in an amount and under conditions such that the antibody, or fragment thereof, inhibits infection.

In accordance with the invention, the antibodies can be administered prior to contact of the subject or the subject's immune system/cells with HIV-1 or after infection of vulnerable cells. Administration prior to contact or shortly thereafter can maximize inhibition of infection of vulnerable cells of the subject (e.g., T-cells).

As indicated above, either the intact antibody or fragment (e.g., antigen binding fragment) thereof can be used in the method of the present invention. Exemplary functional fragments (regions) include scFv, Fv, Fab′, Fab and F(ab′)₂ fragments. Single chain antibodies can also be used. Techniques for preparing suitable fragments and single chain antibodies are well known in the art. (See, for example, U.S. Pat. Nos. 5,855,866; 5,877,289; 5,965,132; 6,093,399; 6,261,535; 6,004,555; 7,417,125 and 7,078,491 and WO 98/45331.)

The antibodies, and fragments thereof, described above can be formulated as a composition (e.g., a pharmaceutical composition). Suitable compositions can comprise the antibody (or antibody fragment) dissolved or dispersed in a pharmaceutically acceptable carrier (e.g., an aqueous medium). The compositions can be sterile and can in an injectable form. The antibodies (and fragments thereof) can also be formulated as a composition appropriate for topical administration to the skin or mucosa. Such compositions can take the form of liquids, ointments, creams, gels, pastes or aerosols. Standard formulation techniques can be used in preparing suitable compositions. The antibodies can be formulated so as to be administered as a post-coital douche or with a condom.

The antibodies and antibody fragments of the invention show their utility for prophylaxis in, for example, the following settings:

i) in the setting of anticipated known exposure to HIV-1 infection, the antibodies described herein (or binding fragments thereof) can be administered prophylactically (e.g., IV or topically) as a microbiocide,

ii) in the setting of known or suspected exposure, such as occurs in the setting of rape victims, or commercial sex workers, or in any sexual transmission with out condom protection, the antibodies described herein (or fragments thereof) can be administered as post-exposure prophylaxis, e.g., IV or topically, and

iii) in the setting of Acute HIV infection (AHI), antibodies described herein (or binding fragments thereof) can be administered as a treatment for AHI to control the initial viral load and preserve the CD4+ T cell pool and prevent CD4+ T cell destruction.

Suitable dose ranges can depend, for example, on the antibody and on the nature of the formulation and route of administration. Optimum doses can be determined by one skilled in the art without undue experimentation. Doses of antibodies in the range of 10 ng to 20 mg/ml can be suitable.

The present invention also includes nucleic acid sequences encoding the antibodies, or fragments thereof, described herein. The nucleic acid sequences can be present in an expression vector operably linked to a promoter. The invention further relates to isolated cells comprising such a vector and to a method of making the antibodies, or fragments thereof, comprising culturing such cells under conditions such that the nucleic acid sequence is expressed and the antibody, or fragment, is produced.

Certain aspects of the invention can be described in greater detail in the non-limiting Example that follows.

### EXAMPLE

### Experimental Details

Study subjects: This study was conducted according to the principles expressed in the Declaration of Helsinki. It was approved by the Institutional Review Boards of the University of Alabama at Birmingham, Rockefeller University, Duke University, and the University of North Carolina. All patients provided written informed consent for the collection of samples and subsequent analysis. Blood samples were obtained from 28 subjects with acute HIV-1 infection and from chronically infected sexual partners of two of them. Blood specimens were generally collected in acid citrate dextrose and plasma separated and stored at −70° C. PBMCs were stored in vapor phase liquid nitrogen.

Laboratory staging: Plasma samples were tested for HIV-1 RNA, p24 antigen, and viral specific antibodies by a battery of commercial tests. These included quantitative Chiron bDNA 3.0 or Roche Amplicor vRNA assays; Coulter or Roche p24 Ag assays; Genetic Systems Anti-HIV-1/2 Plus O; and Genetic Systems HIV-1 Western Blot Kit. Based on these test results, subjects were staged according to the Fiebig classification system for acute and early HIV-1 infection (Fiebig et al, AIDS 17:1871-1879 (2003)).

Viral RNA extraction and cDNA synthesis: For each sample, approximately 20,000 viral RNA copies were extracted using the Qiagen BioRobot EZ1 Workstation with EZ1 Virus Mini Kit v2.0 (Qiagen, Valencia, Calif.). RNA was eluted in 60 ul of elution buffer and subjected to first strand cDNA synthesis immediately by using the SuperScript III Reverse Transcriptase according to manufacturer's instructions (Invitrogen Life Technologies). Each first strand synthesis reaction included ˜10,000 or fewer vRNA molecules, 1× reverse transcription buffer, 0.5 mM of each dNTP, 5 mM DTT, 2 units/ul of RnaseOUT, 10 units/ul of SuperScript III reverse transcriptase and 0.25 uM of antisense primer. The cDNA syntheses were performed using antisense primers located at different genomic regions. The primers for synthesizing the cDNA of env, 5′ half genome (U5, gag and pol) and 3′ half genome (vif, vpr, tat, rev, vpu, env, nef, U3 and R) were env3out 5′-TTGCTACTTGTGATTGCTCCATGT-3′, 1.int.R1 5′-CTTGCCACACAATCATCACCTGCCAT-3′ and 1.R3.B3R 5′-ACTACTTGAAGCACTCAAGGCAAGCTTTATTG-3′, respectively. The reactions were incubated at 50° C. for 60 min, followed by 55° C. for an additional 60 min incubation. The reaction was heat-inactivated at 70° C. for 15 min, and then treated with RNaseH at 37° C. for 20 min. The synthesized cDNA was subjected to 1^{st}round PCR immediately or stored frozen at −80° C.

Proviral DNA Extraction: Blood was collected from subject AD17 14-17 days following exposure to HIV-1 at Fiebig stage II. Genomic DNA was extracted from 1.3 million PBMCs using Qiagen Tissue DNA Extraction kit according to manufacturer's instructions.

Single genome amplification: cDNA or genomic DNA was serially diluted and distributed in replicates of 8 PCR reactions in MicroAmp 96-well plates (Applied Biosystems, Foster City, Calif.) so as to identify a dilution where PCR positive wells constituted less than 30% of total number of the reactions. At this dilution, most wells contain amplicons derived from a single cDNA molecule. Additional PCR amplifications were performed using this dilution in 96-well reaction plates. PCR amplification was carried out in presence of 1× High Fidelity Platinum Taq PCR buffer, 2 mM MgSO4, 0.2 mM each deoxynucleoside triphosphate, 0.2 uM each primer, and 0.025 units/ul of Platinum Taq High Fidelity polymerase in a 20-ul reaction (Invitrogen, Carlsbad, Calif.). The nested primers for generating different genomic segments included: (1) full length env: 1^{st} round sense primer env5out 5′-TAGAGCCCTGGAAGCATCCAGGAAG-3′, 1^{st} round antisense primer env3out 5′-TTGCTACTTGTGATTGCTCCATGT-3′, 2^{nd} round sense primer env5in 5′-TTAGGCATCTCCTATGGCAGGAAGAAG-3′ and 2^{nd} round antisense primer env3in 5′-GTCTCGAGATACTGCTCCCACCC-3′; (2) 5′ half genome: 1^{st} round sense primer 1.U5.F1 5′-CCTTGAGTGCTTCAAGTAGTGTGTGCCCGTCTGT-3′, 1^{st} round antisense primer 1.int.R1 5′-CTTGCCACACAATCATCACCTGCCAT-3′, 2^{nd} round sense primer 2.U5.F2 5′-GTAGTGTGTGCCCGTCTGTTGTGTGACTC-3′ and 2^{nd} round antisense primer 2.int.R2 5′-CAATCATCACCTGCCATCTGTTTTCCATA-3′; (3) 3′ half genome: 1^{st} round sense primer 1.int.F1 5′-ACAGCAGTACAAATGGCAGTATT-3′, 1^{st} round antisense primer 1.R3.B3R 5′-ACTACTTGAAGCACTCAAGGCAAGCTTTATTG-3′, 2^{nd} round sense primer 2.int.F2 5′-TGGAAAGGTGAAGGGGCAGTAGTAATAC-3′ and 2^{nd} round antisense primer 2.R3.B6R 5′-TGAAGCACTCAAGGCAAGCTTTATTGAGGC-3′. PCR parameters were as follows: 94° C. for 2 min, followed by 35 cycles of 94° C. for 15 s, 58° C. for 30 s, and 68° C. for 4 min (env) or 5 min (5′ or 3′ half genomes), followed by a final extension of 68° C. for 10 min. The product of the first-round PCR was subsequently used as a template in the second-round PCR under same conditions but with a total of 45 cycles. The amplicons were inspected on precast 1% agarose E-gel 96 (Invitrogen Life Technologies, Carlsbad, Calif.). All PCR procedures were carried out under PCR clean room conditions using procedural safeguards against sample contamination, including pre-aliquoting of all reagents, use of dedicated equipment, and physical separation of sample processing from pre- and post-PCR amplification steps.

DNA sequencing. Amplicons were directly sequenced by cycle-sequencing using BigDye Terminator chemistry and protocols recommended by the manufacturer (Applied Biosystems, Foster City, Calif.). Sequencing reaction products were analyzed with an ABI 3730x1 genetic analyzer (Applied Biosystems; Foster City, Calif.). Both DNA strands were sequenced using partially overlapping fragments. Individual sequence fragments for each amplicon were assembled and edited using the Sequencher program 4.8 (Gene Codes; Ann Arbor, Mich.). All chromatograms were inspected for sites of mixed bases (double peaks), which would be evidence of priming from more than one template or the introduction of PCR error in early cycles. Any sequence with evidence of double peaks was excluded from further analysis.

Sequence alignments: All the sequence alignments were initially made with Clustal W and then hand-checked using MacClade 4.08 to improve the alignments according to the codon translation. Consensus sequences were generated for each individual. The full sequence alignment is available as a supplemental data file (www.hiv.lanl.gov/content/sequence/HIV/USER_ALIGNMENTS/Li) and sequences are deposited in GenBank (accession numbers:GU330247-GU331770.

Sequence diversity analysis. Complete env sequences (n=1307) were derived from 30 individuals, and 5′ (U5, gag and pol) and 3′ (vif, vpu, tat, rev, env, nef U3, and R) half genome sequences (n=188) were derived from PBMC and plasma at two different time points from subject AD17. Sequences were analyzed for maximum sequence diversity and each set of sequences was then visually inspected using neighbor-joining and Highlighter tools (www.hiv.lanl.gov). Phylogenetic trees were generated by the neighbor-joining method using Clustal W or PAUP.

Hypermutated samples. Enrichment for APOBEC3G/F mutations violates the assumption of constant mutation rate across positions as the editing performed by these enzymes are base and context sensitive. Enrichment for mutations with APOBEC3G/F signatures was assessed using Hypermut 2.0 (www.hiv.lanl.gov). Sequences that yielded a p-value of 0.05 or lower were considered significantly hypermutated and excluded from subsequent analyses.

Proviral DNA cloning. To obtain an infectious molecular clone of the transmitted/founder virus of subject FMS, overlapping 5′ and 3′ half genomes from proviral DNA of earliest sample (day 14) were amplified by single round PCR using Phusion Hot Start High-Fidelity DNA polymerase (Biolabs). Both fragments contained a complete LTR element and an overlap of 170 base pairs encompassing a unique SalI restriction site. For the 5′ half genome, U3-R-U5, gag, pol, vif, vpr and tatI was amplified. For the 3′ half genome, tat1, rev1, vpu, env, nef, tat2, rev2 and U3-R-U5 was amplified. The primers were designed to complement exactly the confirmed transmitted/founder sequence as determined by SGA-direct amplicon sequencing. The recognition sequences of MluI and NotI restriction enzymes were appended to the 5′ ends of the sense and antisense primers, respectively. Single round bulk PCR amplifications were carried out in the presence of 1× Phusion Hot Start HiFi buffer, 0.2 mM of each deoxynucleoside triphosphate, 0.5 uM of each primer, 3% final concentration of DMSO, and 0.02 units/ul of Phusion Hot Start High Fidility polymerase in 50 ul reactions. The PCR product of each half genome was subjected to MluI and NotI digestion and gel purification and then independently cloned into the MluI-NotI site of TOPO XL vector (Invitrogen). The ligation mixture was transformed into XL2 Blue MRF competent cells and plated onto LB agar plates supplemented with 50 ug/ml of kanamycin and grown overnight at 30° C. Single colonies were selected and grown overnight in LB medium with same concentration of kanamycin at 30° C. with constant shaking. Plasmid DNA was isolated and sequenced to confirm the identity of transmitted/founder sequences. The 5′ genome half was excised and cloned into 3′ TOPO XL vector by utilizing the MluI and SalI restriction sites thereby generating the full length clone of the transmitted/founder provirus.

Phenotypic analyses. Replication competency of the full length molecular proviral clone AD17.1 was assessed using 293T cells, JC53BL-13 cells (NIH AIDS Research and Reference Reagent Program catalogue #8129, TZM-bl), activated primary human CD4+ lymphocytes, and monocyte-derived macrophages. Infectious virus stock generation, Env-pseudotyped virus stocks, titrations, cell infections and virus neutralization assays were performed according to methods previously described (Salazar-Gonzalez et al, J. Virol. 82:3952-3970 (2008)). Virus controls (replication competent or Env-pseudotyped) included the HIV-1 macrophage-tropic strains YU2 and BaL, the non-macrophage tropic T-cell line-adapted strain NL4.3, the dual R5/X4 tropic stain WEAU1.60, and the xenotropic MuLV env (Salazar-Gonzalez et al, J. Virol. 82:3952-3970 (2008)). The coreceptor inhibitors TAK779 and AMD3100 were obtained from the NIH AIDS Research and Reference Reagent Program (4983 and 8128). R5 and X4 tropism was assessed in both JC53BL-13 cells and in GHOST(3) cells that stably express CD4 along with CCR5 or CXCR4 or both or neither coreceptor.

Recombination analyses. Recombination was evaluated using GARD (Kosakovsky et al, Bioinformatics 22:3096-3098 (2006)) and Recco (Maydt et al, Bioinformatics 22:1064-1071 (2006)) and by visual inspection of Highlighter plots. The minimum number of recombination events required to explain sequence datasets was estimated using the four-gamete method of Hudson and Kaplan (Hudson et al, Genetics 111:147-164 (1985)) as implemented in DNASP v5.00.07 (Rozas et al, Bioinformatics 19:2496-2497 (2003)). Recombinant sequences reported in Table 2 were identified by Highlighter analysis and confirmed by Hudson-Kaplan, GARD and Recco analyses.

Mathematical model. The model employed in the present study has been described (Keele et al, Proc. Natl. Acad. Sci. USA 105:7552-7557 (2008), Salazar-Gonzalez et al, J. Exp. Med. 205:1273-1289 (2009), Lee et al, J. Theor. Biol. 261:341-360 (2009)) as have measured parameters of early virus expansion (Little et al, J. Exp. Med. 190:841-850 (1999), Stafford et al, J. Theor. Biol. 203:285-301 (2000), Markowitz et al, J. Virol. 77:5037-5038 (2003)). Under this model, with no selection pressure and fast expansion, one can expect small samples from homogeneous virus populations to have evolved from a founder strain in a star-like phylogeny with all sequences coalescing at the founder (Keele et al, Proc. Natl. Acad. Sci. USA 105:7552-7557 (2008), Keele et al, J. Exp. Med. 206:1117-1134 (2009)). Occasional deviations from a star phylogeny are, however, expected. The sampling of 10 sequences, for example, from a later generation of an exponentially growing population with six-fold growth per generation (R₀=6) has about 3% chance of including at least one pair sharing the first four generations, a 19% chance of including sequences that share three, and a 75% chance of sharing two. Using a point mutation rate of about 1 per 5 generations for the full-length 9 kb HIV-1 genome (Keele et al, Proc. Natl. Acad. Sci. USA 105:7552-7557 (2008), Lee et al, J. Theor. Biol. 261:341-360 (2009)), there is about 75% chance of finding among ten sequences two that share one mutation, about 20% chance of finding two sequences that share a pair of mutations, and <2% chance of sharing more than that. These probabilities are slightly enhanced by early stochastic events that can lead to the virus producing less than six descendants in some generations but are diminished by the chances that mutations cause a fitness disadvantage that results in early purifying selection, as previously observed (Keele et al, Proc. Natl. Acad. Sci. USA 105:7552-7557 (2008), Wood et al, PLoS Pathog. 5:el 000414 (2009)). Examples of such early stochastic mutations leading to deviations from star phylogeny were found in several subjects (Table 2).

Statistical analyses and power calculations. A calculation was made of the statistical significance of differences in rates of single versus multivariant HIV-1 transmission using Fisher's exact test. Differences were considered statistically significant at a value of p≦0.05. To estimate the likelihood of missing infrequently represented transmitted variants, a power study was described previously that estimated the probability of sampling low frequency plasma viral sequences (Keele et al, Proc. Natl. Acad. Sci. USA 105:7552-7557 (2008)). In a sample set of at least n=20, there is a 95% probability that a given missed variant comprises less than 14% of the virus population. For a sample set of 30, there is a 95% probability not to miss a variant that comprises at least 10% of the total viral population. And for a sample set of 80, there is a 95% probability not to miss a variant that comprises at least 4% of the total viral population.

Also considered was the possibility that the number of transmitted/founder viruses detected could be influenced by the clinical stage (Fiebig stage) of the subjects at the time of virus sampling, because differences in virus replication rates could lead to increasing differences in virus frequencies with time. If this were the case and some viruses were outcompeted, the prediction would be that at later Fiebig stages the numbers of transmitted/founder virus lineages would be less than at earlier Fiebig stages. The model (which is based on previously estimated parameters of an HIV-1 generation time of 2 days, a reproductive ratio [R₀] of 6, and a reverse transcriptase error rate of 2.16×10⁻⁵ and assumes that the initial virus replicates exponentially infecting R₀ new cells at each generation and diversifies under a model of evolution that assumes no selection) predicts that descendants of a transmitted virus at 45% replicative disadvantage compared to another transmitted virus, still have more than a 5% chance of occurring in a sample size of 20, ten generations (˜20 days) later. In humans, the eclipse period, defined as the time between HIV-1 transmission and first detection of virus in the plasma, has been estimated to be approximately 10-14 days, and the eclipse period plus Fiebig stages I and II, approximately 22-26 days (Keele et al, Proc. Natl. Acad. Sci. USA 105:7552-7557 (2008), Fiebig et al, AIDS 17:1871-1879 (2003)}. In the present study, the numbers of subjects in Fiebig stages I/II, III, IV and V were 14, 2, 6 and 6, respectively, and this relative distribution was similar in the three other studies included in the combined analysis (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008)). As described in the main text, there was no significant correlation between clinical stage and multiplicity of infection (Fisher's exact p=0.53).

### Results

Study Subjects. SGA-direct sequencing was used to identify and enumerate transmitted/founder env sequences in 28 acutely infected MSM subjects who reported homosexual exposure as their primary HIV-1 risk behavior and who denied injection drug use (Table 1). At the time of study, 14 subjects were HIV-1 ELISA negative/western immunoblot (WB) negative (Fiebig stage II), 2 were ELISA+/WB− (Fiebig stage III), 6 were ELISA+/WB indeterminate (Fiebig stage IV) and 6 were ELISA+/WB+/p31− (Fiebig stage V) (Keele et al, Proc. Natl. Acad. Sci. USA 105:7552-7557 (2008), Fiebig et al, AIDS 17:1871-1879 (2003)). Subjects were identified based on clinical symptoms of an acute retroviral syndrome, routine HIV testing in a health care setting, or contact tracing of an HIV-1 infected index case. Clinical histories of sexually transmitted diseases were not available. Envelope sequences from chronically infected sexual partners of two acutely infected subjects were also evaluated.

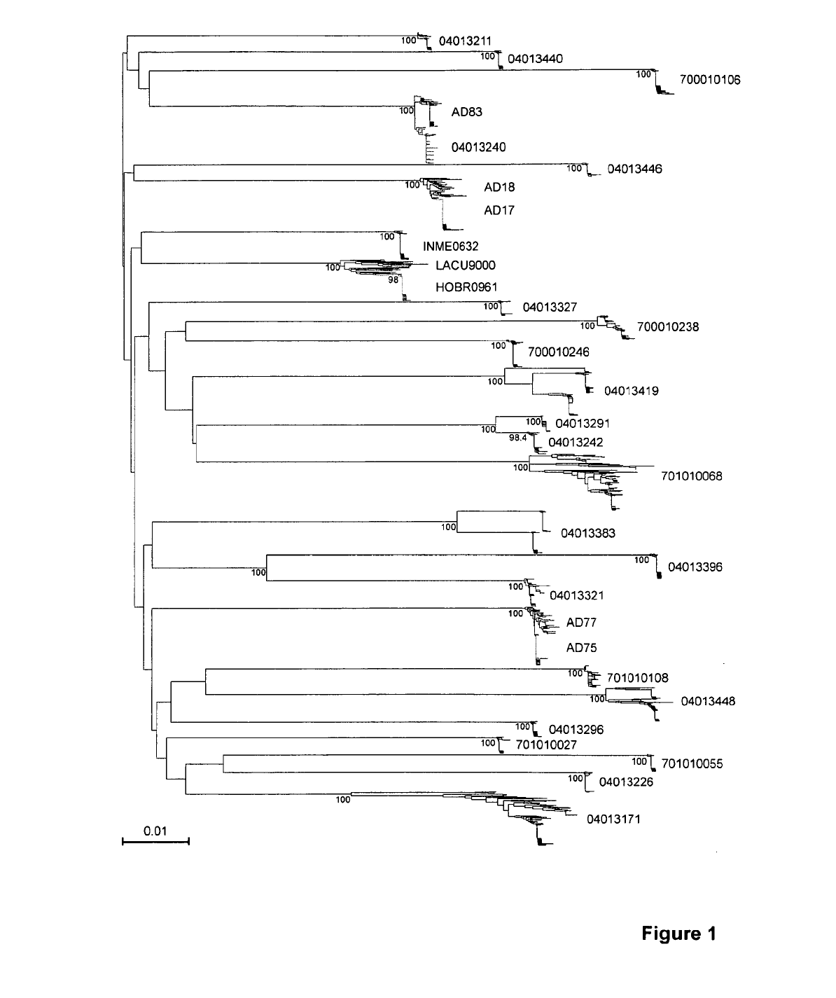

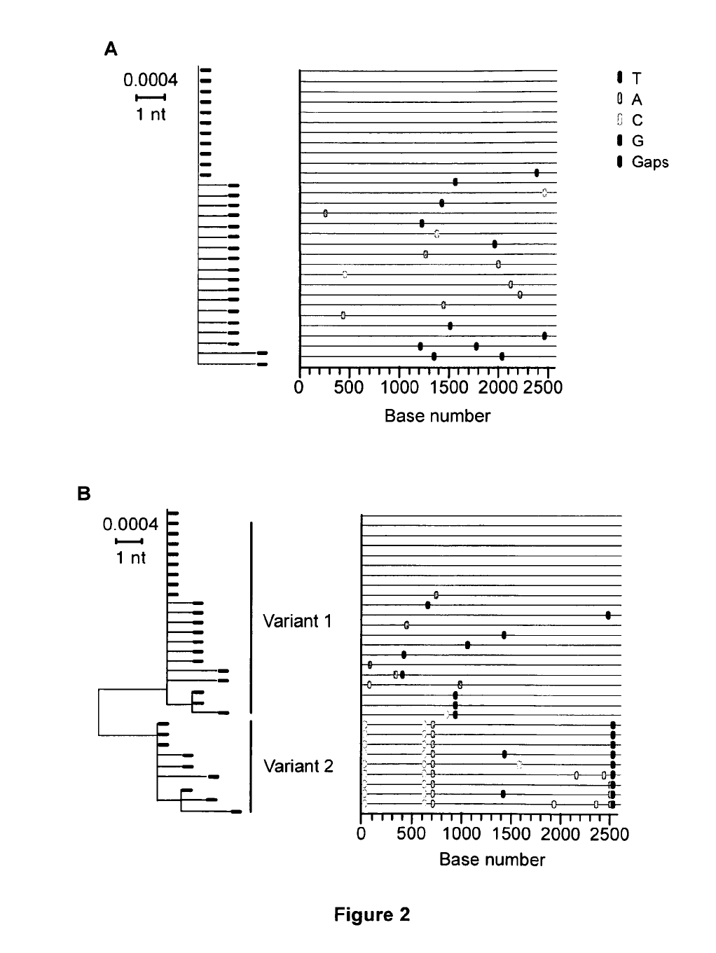

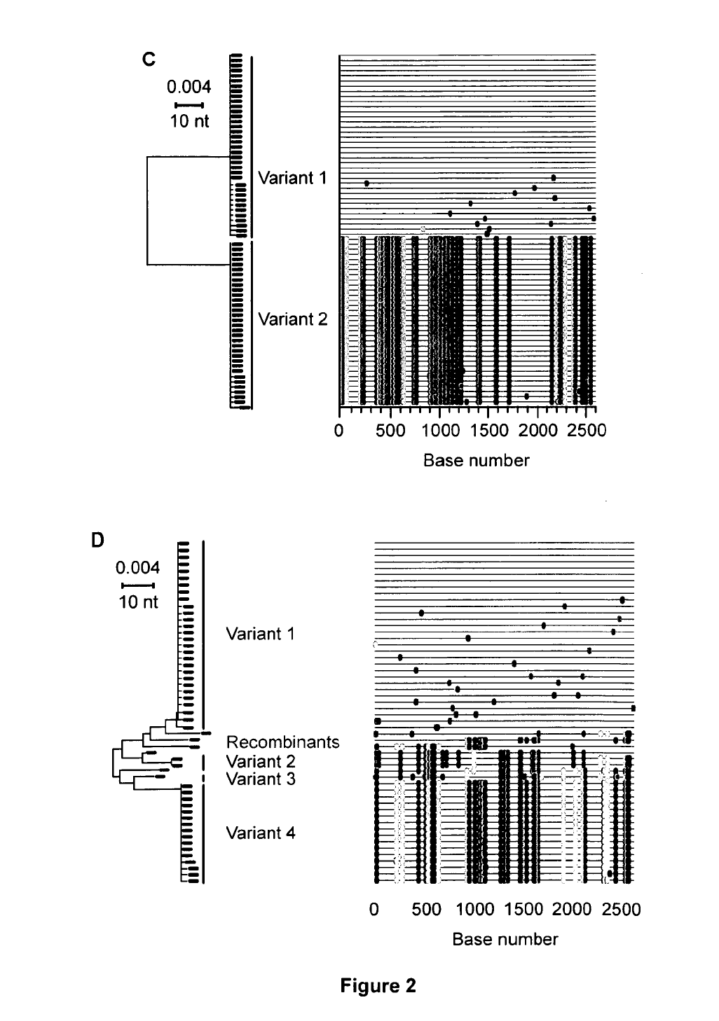

HIV-1 Env Diversity Analysis. A total of 1307 full-length env genes encoding gp160 were sequenced from plasma vRNA (median of 40 sequences per subject; range 23-89). In a composite neighbor-joining (NJ) phylogenetic tree (FIG. 1), viral sequences formed distinct patient-specific monophyletic lineages, each with high statistical support. Sequences from known sexual partners, including two acute-to-acute (AD77 to AD75 and AD83 to 04013240) and two chronic-to-acute (LACU9000 to HOBRO961 and AD18 to AD17) transmission pairs, also clustered significantly together (FIG. 1). All sequences were HIV-1 subtype B. Among the 28 acutely infected subjects, maximum within-patient env diversities ranged from 0.12% to 6.82% (Table 2). Sequences from 22 of these individuals had distinctly lower env diversities (<0.75%) compared with env diversities from six others (>1.25%). The latter diversity is inconsistent with single virus transmission within the time frame of acute and early infection (Fiebig stage I-V) (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008), Lee et al, J. Theor. Biol. 261:341-360 (2009)), while env diversity <0.75% is consistent either with single variant transmission or with transmission of two or more closely related viruses. Phylogenetic and Highlighter analyses of env sequences distinguished between these possibilities for each subject (FIG. 2; see also www.hiv.lanl.gov/content/sequence/HIV/USER_ALIGNMENTS/Li). FIG. 2A shows sequences from a subject (04013440) who was infected by a single virus, FIG. 2B a subject (04013211) infected by two viruses differing by only 4 nucleotides out of 2619 (0.15%), FIG. 2C a subject (04013383) infected by two viruses differing by 65 of 2547 nucleotides (2.55%), and FIG. 2D a subject (04013448) infected by four viruses differing by as many as 47 of 2655 nucleotides (1.79%) with additional sequences showing recombination between the transmitted/founder lineages. Altogether, it was determined that 10 of 28 subjects (36%) had been productively infected by more than one virus (Table 2).

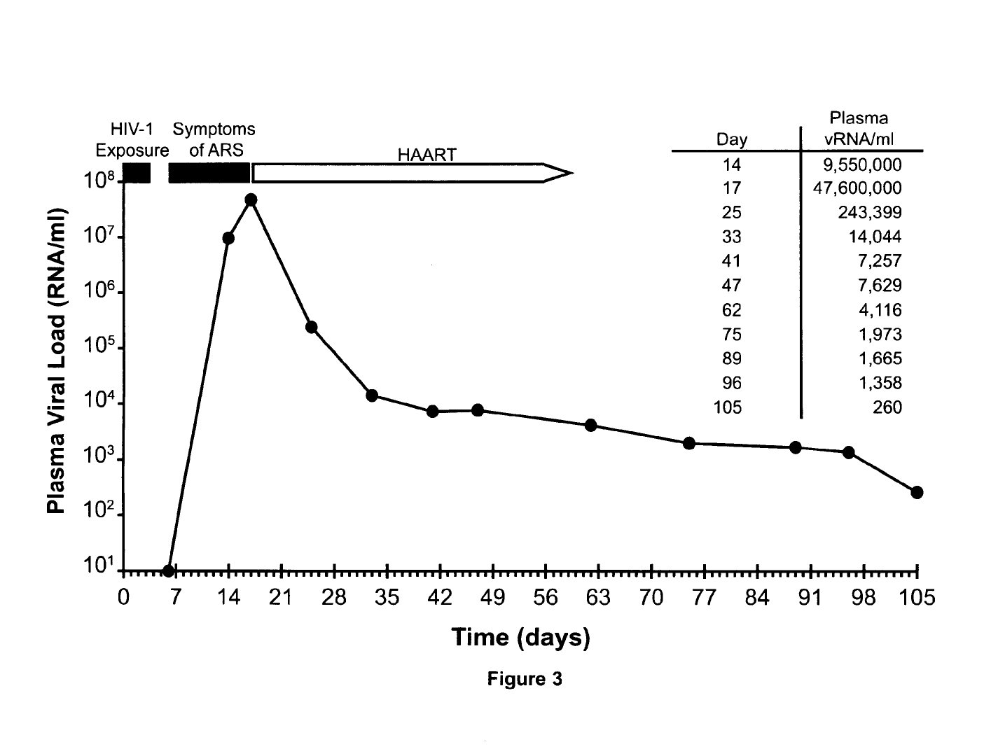

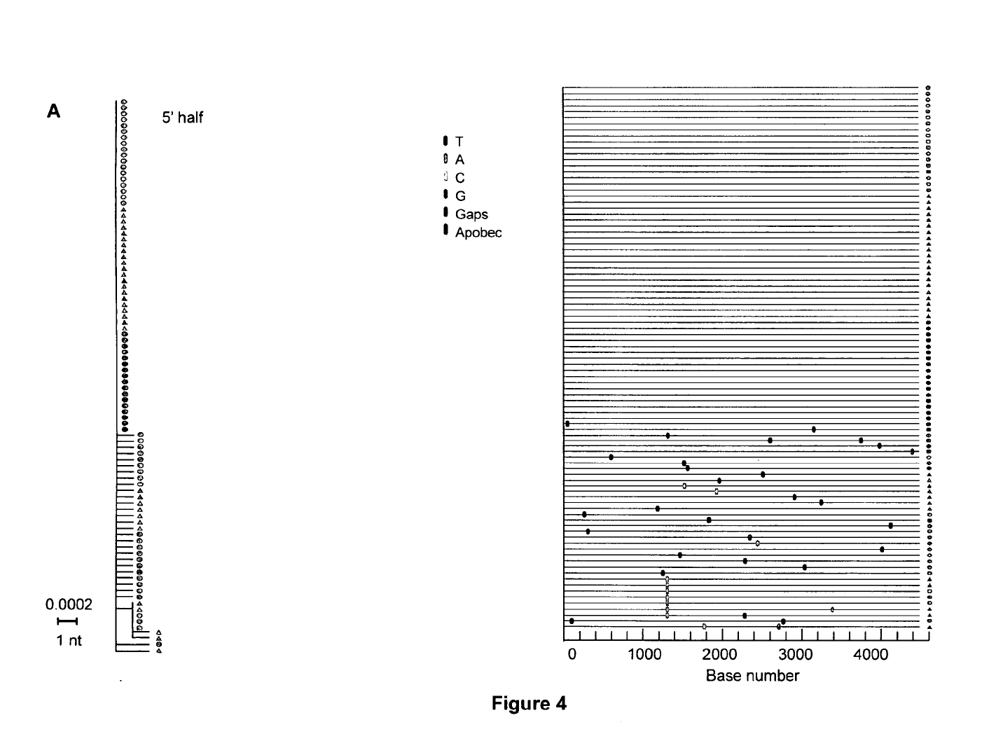

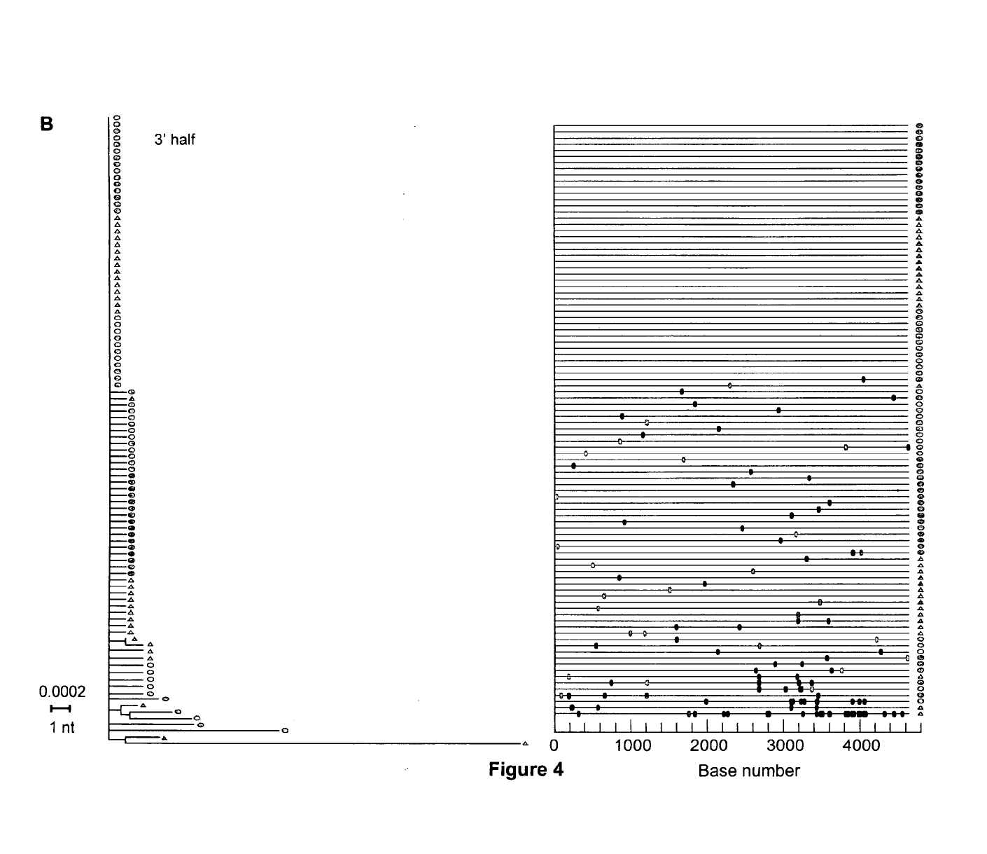

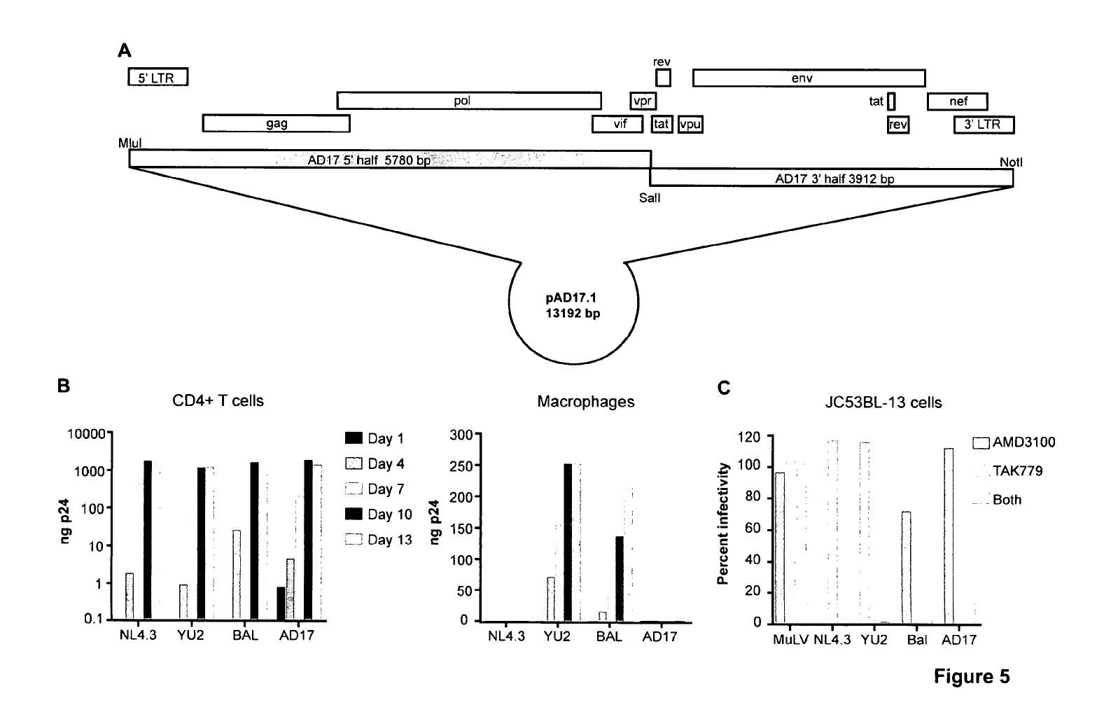

Model Analysis of HIV-1 Diversification. The env sequences were next analyzed using a mathematical model of random virus evolution described previously (Keele et al, Proc. Natl. Acad. Sci. USA 105:7552-7557 (2008), Keele et al, J. Exp. Med. 206:1117-1134 (2009), Lee et al, J. Theor. Biol. 261:341-360 (2009)). Sequences resulting from multivariant transmission, APOBEC hypermutation, early stochastic mutation, selection by cytotoxic T-cells, or recombination violate model predictions (Table 2) (Keele et al, Proc. Natl. Acad. Sci. USA 105:7552-7557 (2008), Lee et al, J. Theor. Biol. 261:341-360 (2009)). Once these confounders were accounted for, lineage-specific env sequences from each subject conformed to model predictions and coalesced to most recent common ancestor sequences at or near the time of virus transmission estimated from clinical histories and laboratory staging. These results thus corroborated a large body of evidence supporting the SGA-direct sequencing strategy for identifying transmitted/founder viruses (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008), Salazar-Gonzalez et al, J. Virol. 82:3952-3970 (2008), Salazar-Gonzalez et al, J. Exp. Med. 205:1273-1289 (2009), Keele et al, J. Exp. Med. 206:1117-1134 (2009), Kearney et al, J. Virol. 83:2715-2727 (2009)). As an additional test of the model's validity, it was asked in subject AD17, whose history of virus exposure was particularly well-documented (Table 1 and FIG. 3), if plasma vRNA and PBMC viral DNA (vDNA) sequences spanning the complete (9.2 Kb) viral genome coalesced to the same viral sequence as did env-only sequences and if a molecular clone of this viral genome encoded replication competent virus, as would be expected for an authentic transmitted/founder virus. For this analysis, SGA-direct sequencing was used to determine env-only sequences (n=51) and overlapping 5′ (n=92) and 3′ (n=96) half genome sequences (FIG. 4). All 239 vRNA and vDNA sequences coalesced to a single transmitted/founder genome in a time frame consistent with the clinical history of virus exposure as recently as 11 days earlier. The estimated time to a most recent common ancestor (MRCA) sequence for env-only sequences was 8 days (95% CI 5-11) and for all sequences 6-11 days (CI 3-14). MRCA estimates are frequently lower than clinical estimates (Keele et al, Proc. Natl. Acad. Sci. USA 105:7552-7557 (2008)) or experimentally determined intervals between transmission and virus sampling in the rhesus macaque-SIV infection model (Keele et al, J. Exp. Med. 206:1117-1134 (2009)) because of purifying selection or variance in estimated parameters of virus replication (Lee et al, J. Theor. Biol. 261:341-360 (2009)). The inferred transmitted/founder viral genome in subject AD 17 contained intact LTR-gag-pol-vif-vpr-tat-rev-vpu-env-nef-LTR elements, a finding replicated for transmitted/founder viruses from 38 other subjects infected by HIV-1 subtypes A, B, C or D (Salazar-Gonzalez et al, J. Exp. Med. 206:1273-89(2009)). A proviral clone (pAD 17.1) of the transmitted/founder viral genome from subject AD17 (FIG. 5A), when transfected into 293T cells, produced virions that were infectious and highly replicative in human CD4+ T-cells but, interestingly, not in monocyte-derived macrophages from the same normal donors (FIG. 5B). pAD17.1 virus was CCR5 tropic in JC53BL-13 cells (FIG. 5C) and in GHOST(3) cells (Morner et al, J. Virol. 73:2343-49 (1999)), where it infected cells bearing CD4 and CCR5 but not CD4 and CXCR4).

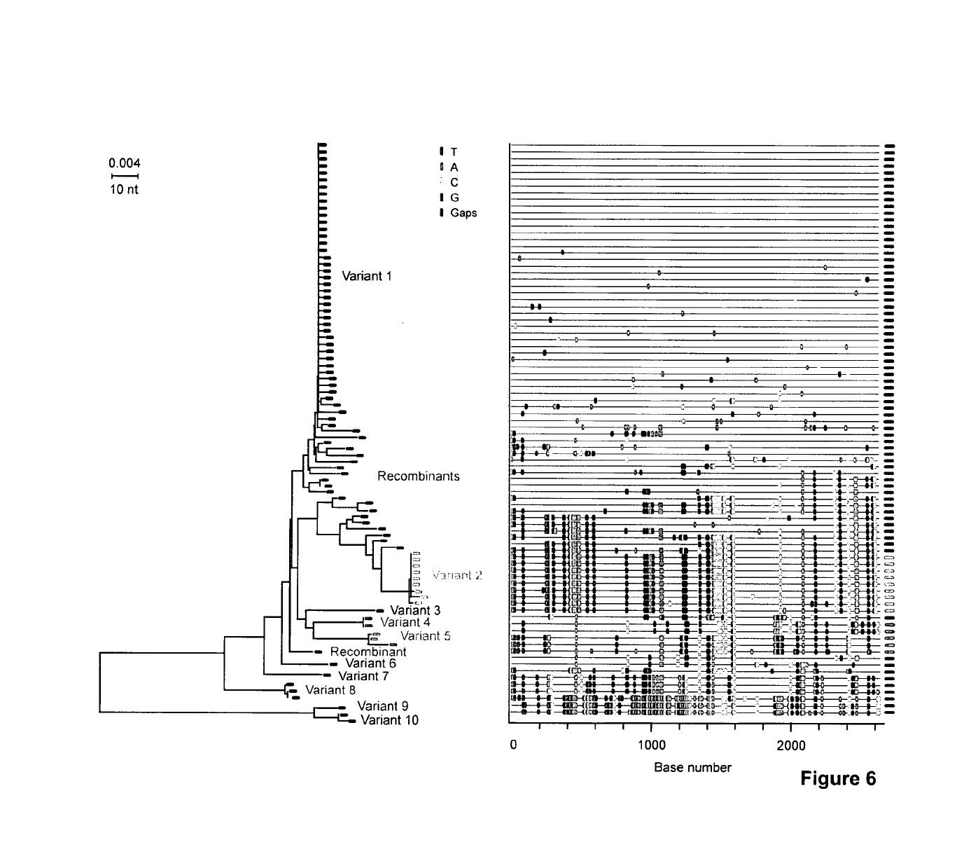

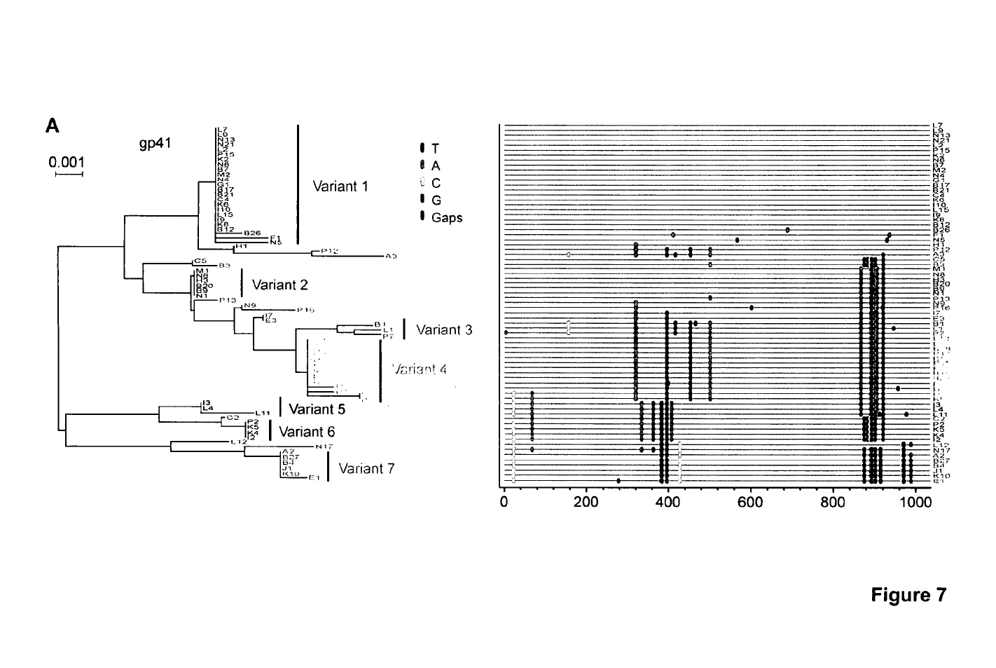

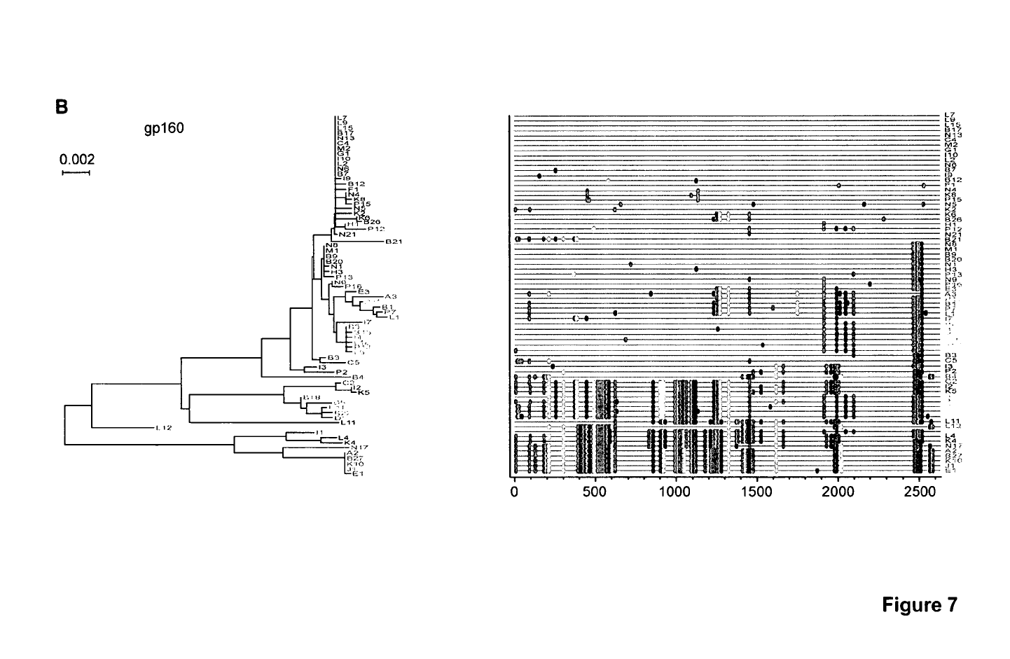

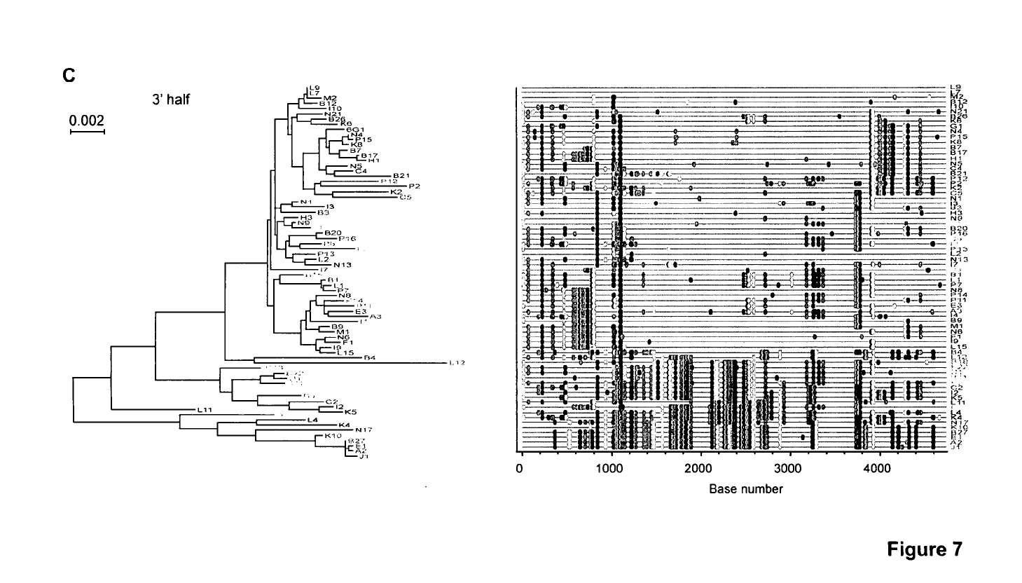

High Multiplicity Infection Followed by Recombination. Extremes in HIV-I diversity in acute infection could be informative regarding biological events underlying virus transmission. Subject 04013171 had the greatest env diversity (6.82%) (Table 2). This subject admitted to unprotected receptive anal intercourse with multiple partners over a single eight hour period four weeks before the onset of flu-like symptoms, consistent with his Fiebig IV staging. FIG. 6 shows a NJ tree and Highlighter plot of 86 plasma derived env sequences, which revealed 10 unique transmitted/founder virus lineages. In addition, 20 inter-lineage recombinants were identified based on shared polymorphisms in the Highlighter plot with corroboration by Recco analysis (Maydt et al, Bioinformatics 22:1064-71 (2006)). Among these recombinant sequences, the Hudson-Kaplan test (Hudson et al, Genetics 111:147-164 (1985)) indicated a minimum of 44 recombination breakpoints. Interestingly, sequences corresponding to 4 of the 10 virus lineages in subject 04013171 were sampled only once. It was possible to be confident that these represented a unique transmitted/founder viruses and not recombinants between two or more predominant virus lineages because of the large number of unique nucleotide changes in each sequence that far exceeded the diversity observed empirically(Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008), Keele et al, J. Exp. Med. 206:1117-1134 (2009)) or estimated to occur based on mathematical modeling (Keele et al, Proc. Natl. Acad. Sci. USA 105:7552-7557 (2008)) in the first 35 days of infection (eclipse phase to the end of Fiebig stage IV) (Keele et al, Proc. Natl. Acad. Sci. USA 105:7552-7 (2008)). Power calculations further indicated that with a sample size of 86 sequences, there is a >95% probability of detecting minor sequences representing at least 4% of the population (see Experimental Details). These findings suggest that more extensive sampling might result in the detection of an even greater number of transmitted/founder viruses in this individual. Subject 701010068 had the second highest env diversity (4.43%) among the study subjects (Table 2). He reported a single high risk exposure event involving unprotected receptive anal intercourse with two individuals, one HIV negative and the other HIV positive. He developed flu-like symptoms approximately two weeks later and was studied three weeks after that, again at Fiebig stage IV. Based on the earlier analysis of subject 04013171 (FIG. 6), there was a concern that viral recombination (Jung et al, Nature 418:144 (2002), Levy et al, Proc. Natl. Acad. Sci. USA 101:4204-4209 (2004), Shriner et al, Genetics 167:1573-1583 (2004), Simon-Loriere et al, PLoS Pathog. 5:e1000418 (2009)), which is sequence length and time (from infection) dependent (Keele et al, Proc. Natl. Acad. Sci. USA 105:7552-7557 (2008), Keele et al, J. Exp. Med. 206:1117-1134 (2009), Levy et al, Proc. Natl. Acad. Sci. USA 101:4204-4209 (2004)), could confound the identification of discrete transmitted/founder virus lineages. This could be especially problematic in subjects infected with many different transmitted/founder viruses as opposed to two, since in the former case it is far more likely that doubly or multiply infected cells will spawn heterozygous virus progeny that lead in the next virus generation to recombinant viral genomes (Jung et al, Nature 418:144 (2002), Levy et al, Proc. Natl. Acad. Sci. USA 101:4204-4209 (2004), Shriner et al, Genetics 167:1573-1583 (2004)). To test this hypothesis, seventy-two 3′ half genome segments of plasma vRNA from subject 701010068 were amplified and sequenced and then env gp41 (1035 bp), env gp160 (2630 bp), and 3′ half genome regions (4734 bp) were analyzed separately. The gp41 sequences (FIG. 7A) revealed discrete low diversity lineages comprised of identical or nearly identical sequences. Seven of these sequence clusters were interpreted as likely to have arisen from distinct transmitted viruses and the remaining sequences to represent inter-lineage recombinants. Clusters of identical or nearly identical sequences were also evident in gp160 sequences (FIG. 7B), but with less clarity due to additional inter-lineage recombination events in the longer sequences. For example, sequences corresponding to lineage 4 in the gp41 sequences (depicted in light blue in FIG. 7A) were dispersed into five widely separated branches in the gp160 tree due entirely to recombination (FIG. 3B). Similarly, sequences comprising lineage 6 in the gp41 sequences (depicted in red in FIG. 7A) were dispersed into three widely separated branches in the gp160 tree, again due entirely to recombination (FIG. 7B). These findings were supported by the Hudson-Kaplan analysis (Hudson et al, Genetics 111:147-164 (1985)), which indicated a minimum of 27 recombination breakpoints among the gp160 env sequences. Interspersion of sequences was even more dramatic in the 3′ half genome tree (FIG. 7C). Remarkably, of the 72 3′ half genome sequences depicted in FIG. 7C, 63 (88%) represented overt recombinants between two or more transmitted/founder lineages demonstrable by visual inspection and by computer-assisted algorithms. Only two (dark blue) sequences labeled L7 and L9 at the very top of the tree (FIG. 7C), three (green) sequences labeled B1, L1 and P7 in the middle of the tree, and four (gray) sequences labeled B27, E1, A2 and J1 at the very bottom of the tree showed no evidence of recombination. These findings, together with Corroborating data from env-only sequences (Abrahams et al, J. Virol. 83:3556-3567 (2009), Keele et al, Proc. Natl. Acad. Sci. USA 105:7552-7557 (2008), Salazar-Gonzalez et al, J. Virol. 82:3952-3970 (2008)), lead to the surprising conclusion that by the time of first antibody detection in acute HIV-1 infection (Fiebig stages III/IV), a majority of circulating viruses may be recombinants. This finding is testament to the large number of doubly (or multiply) infected cells in acute and early infection and further evidence of the rapidity with which virus diversifies (Bimber et al, J. Virol. 83:8247-8253 (2009), Goonetilleke et al, J. Exp. Med. 206:1253-1272 (2009)), making clear that in order to identify non-recombinant transmitted/founder HIV-1 (or SIV) genomes (Keele et al, Proc. Natl. Acad. Sci. USA 105:7552-7557 (2008), Salazar-Gonzalez et al, J. Exp. Med. 205:1273-1289 (2009), Keele et al, J. Exp. Med. 206:1117-1134 (2009), it is necessary to characterize viral sequences as close to the transmission event as possible.

Comparisons of Multiplicity of HIV-1 Infection in MSM versus Heterosexuals. Four studies, including the present one, have estimated the numbers of viruses responsible for transmission and productive HIV-1 infection after heterosexual or homosexual exposure using identical SGA-direct env amplicon sequencing methods (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008)). One of these evaluated the frequency of multivariant transmission in a cohort of cohabitating HIV-1 discordant (antiretroviral naïve) heterosexual couples in Zambia and Rwanda followed prospectively for HIV-1 transmission (Haaland et al, PLoS Pathog. 5:e1000274 (2009)). Remarkably, only 2 of 20 [10%; 95% confidence interval (CI) 1-32%] of the epidemiologically linked infections resulted from multivariant transmission, a finding attributed to the chronicity of infection in the virus positive partner, lower prevelance of comorbid conditions such as untreated tuberculosis or sexually transmitted infections, and the heterosexual route of transmission. Since the frequency of multivariant transmission in HSX in that study was substantially less than what was observed for MSM [2 of 20 HSX (10%, CI 1-32%) versus 10 of 28 MSM (36%, CI 19-56%); Fisher's exact p=0.042, odds ratio 4.85, 95% CI 1.1-inf], a combined analysis was performed of data from all four studies, which included 225 patients infected by HIV-1 subtypes A, B or C (Table 3). Again, it was found that the proportion of MSM subjects infected by more than one virus was substantially higher than for HSX [19 of 50 (38%) versus 34 of 175 (19%); Fisher's exact p =0.008, odds ratio 2.5, 95% CI 1.2-5.3]. The MSM subjects were all infected with subtype B; a comparison to only the subset of HSX infections that were subtype B was still significant (Fisher's exact p=0.01, odds ratio 2.9, 95% CI 1.2-7.1). The frequency of multiple infections in HSX was not statistically different among subtypes A, B and C nor was it different between males and females. Differences in the frequency of multivariant HIV-1 transmission in MSM versus HSX could not be accounted for by the numbers of sequences analyzed per subject nor by the clinical stage of subjects at the time of study. In the study by Haaland et al (PLoS Pathog. 5:e1000274 (2009)) the median number of sequences analyzed was 40, in Keele et al (Proc. Natl. Acad. Sci. USA 105:7552-7557 (2008)) it was 25, and in Abrahams et al (J. Virol. 83:3556-3567 (2009)), it was 22. In the present study, the median number of sequences that were determined as part of the initial survey was 33 (Table 2). This lower number of sequences was used for statistical comparisons of single and multivariant transmissions in MSM versus HSX subjects in order to allow for comparability among the four studies. When, in this initial sequence set, samples were identified containing more than one transmitted/founder virus lineage, additional sequences (as many as 89) were then obtained in order to estimate more precisely the numbers of transmitted/founder viruses (Table 2). Increasing the numbers of sequences analyzed allowed for greater accuracy and precision in estimating the numbers of viruses transmitted in those individuals with many transmitted viruses (e.g., subjects 4013448, 04013171 and 701010068 in FIGS. 2D, 6 and 7), but it did not affect the discrimination between those subjects infected by one virus versus those infected by more than one virus. Finally, no significant correlation was found between the clinical stage of subjects at the time of plasma sampling and the numbers of transmitted/founder viruses identified in those samples: among the four studies, there was a total of 95 antibody negative subjects (Fiebig stages I-II) and 130 antibody positive subjects (Fiebig stages III-VI). Twenty subjects (21%) in the former group and 33 subjects (25%) in the latter group had evidence of productive infection by more than one virus, which was not significantly different (odds ratio 1.27; 95% CI 0.65-2.54; Fisher's exact p=0.53).

Previous studies used less precise methods for estimating multiplicity of HIV-1 infection in HSX and MSM subjects and reported widely varying results with a trend for higher multiplicities in MSM (Gottlieb et al, J. Infect. Dis. 197:1011-1015 (2008), Grobler et al, J. Infect. Dis. 190:1355-1359 (2004), Learn et al, J. Virol. 76:11953-11959 (2002), Long et al, Nat. Med. 6:71-75 (2000), Poss et al, J. Virol. 69:8118-8122 (1995), Ritola et al, J. Virol. 78:11208-11218 (2004), Sagar et al, J. Virol. 78:7279-7283 (2004), Wolfs et al, Virology 189:103-110 (1992), Zhu et al, Science 261:1179-1181 (1993)). Described here are new SGA-based determinations that show significant differences in the multiplicity of virus infection between the two risk groups: MSM were twice as likely as HSX subjects to become infected by more than one virus, with some MSM acquiring as many as 7 to 10 or more viruses. These findings are consistent with the higher epidemiological risk of HIV-1 acquisition in MSM compared with HSX and may be explained in part by the anatomical and immunohistological differences between the male and female genitourinary tracts and the lower intestine.

A limitation of the current study is that it represents a retrospective comparison of multivariant HIV-1 transmission among historical patient cohorts having different enrollment criteria and different behavioral risk assessments. It must be noted, however, that all study subjects from all cohorts were queried extensively with regard to potential HIV-1 infection risk behaviors. This included acutely infected subjects identified by cross-sectional screening methods (Pilcher et al, N. Engl. J. Med. 352:1873-83 (2005)), subjects enrolled prospectively into HIV-1 discordant couple (Haaland et al, PLoS Pathog 5:e1000274 (2009)) or Acute Infection Early Disease Research Program cohorts (Mehandru et al, J. Virol. 81:599-612 (2007)), and source plasma donors who became HIV-1 infected during a period of serial plasma donations (Keele et al, Proc. Natl. Acad. Sci. USA 105:7552-57 (2008)). The latter subjects, who were studied anonymously, underwent exhaustive pre-enrollment interrogation for HIV and injection drug use risk behaviors according to a standardized FDA-approved protocol (http://wwwfda.gov/BiologicsBloodVaccines/GuidanceComplianceRegulatoryInf ormation/Guidances/Blood/ucm073445.htm) that included a written questionnaire and interview inquiring about MSM and IDU activities, sex-for-money, sex with a partner who had sex-for-money, or sex with an individual known to be HIV positive. Source plasma donors also underwent serial laboratory testing for surrogate laboratory markers that could indicate injection drug use (e.g., liver transaminase elevations and hepatitis B or C nucleic acids or antibodies), and these markers were uniformly negative among qualified donors. Thus, within the limitations of self-reporting and surrogate marker testing, it was possible to be confident that study subjects in the four cohorts examined were correctly assigned to HSX and MSM risk groups and that injection drug use was unlikely. Future studies can benefit from a prospective trial design and a common behavioral and medical questionnaire (Boily et al, Lancet Infect. Dis. 8:200-7 (2008), Boily et al, Lancet Infect. Dis. 9:118-29 (2008)).

It is noteworthy that while multivariant HIV-1 transmission was twice as common in MSM than in HSX, still more than half of MSM subjects showed evidence of productive infection by just one virus. Moreover, the adjusted median (calculated from subjects with multivariant transmissions only) was 3 in MSM compared with 2 in HSX (Table 3). Even in the Fiebig II subject AD17, where a total of 239 sequences were analyzed (giving us a 95% probability of detecting a second transmitted/founder virus lineage at 1.25% prevalence), all of the sequences coalesced phylogenetically to a single virus, thus providing no evidence for transmission of more than one virus. Elsewhere, 454 deep sequencing has been used to analyze tens of thousands of sequences from three additional Fiebig stage II MSM subjects in whom SGA-direct sequencing suggested transmission and productive clinical infection by a single virus. Even with this greatly enhanced sensitivity of detection of minor sequences, we found no evidence of transmission by more than one virus in these subjects. Considered together, the findings of the present study, previously published studies (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008)) and work in progress, all suggest that a substantial proportion of HSX and MSM patients acquire HIV-1 infection as a consequence of transmission and productive infection by literally one virion or one infected cell. The implication of this finding is that in order for a vaccine, microbicide or other prevention modality to be protective in this fraction of individuals, it need only prevent infection by a single virus or infected cell. Conversely, there is another subset of HSX and MSM subjects in whom the multiplicity of infection is higher. Since the proportion of such multiply individuals is far higher than would be expected from a Poisson distribution of independent, low frequency events (see Abrahams et al (J. Virol. 83:3556-3567 (2009)) for discussion), it is suspected that the biological events underlying virus transmission in these subjects compared with those infected by a single virus are different and that challenges faced by vaccines and microbicides in the higher multiplicity infection group may be higher.

Another interesting observation from the present study relates to viral recombination. Although recombination was not a primary study objective, the identification of two or more transmitted/founder genomes in acutely infected subjects provided a unique opportunity to examine the dynamics and extent of recombination in primary HIV-1 infection. Five features of this study distinguish it from previous reports of HIV-1 recombination (Jung et al, Nature 418:144 (2002), Levy et al, Proc. Natl. Acad. Sci. USA 101:4204-4209 (2004), Shriner et al, Genetics 167:1573-1583 (2004), Simon-Loriere et al, PLoS Pathog. 5:e1000418 (2009)). First, subjects were studies very early clinical stages following virus transmission (Fiebig stages II-V). Second, SGA-direct amplicon sequencing was used, which provides for a proportional representation of virus present in the plasma, including those that are recombinant (Salazar-Gonzalez et al, J. Virol. 82:3952-3970 (2008)). Third, SGA eliminates in vitro recombination artifacts resulting from Taq polymerase-mediated template switching (Salazar-Gonzalez et al, J. Virol. 82:3952-3970 (2008)). Fourth, SGA made it possible to identify the exact nucleotide sequences of full-length transmitted/founder virus env genes unambiguously and to distinguish these viruses and their progeny from viruses that contained even short regions of recombinant sequence. Fifth, SGA-direct sequencing of a 3′ half genomes made it possible to examine recombination across the boundaries of vif-vpr-tat-rev-vpu-env-nef-LTR. FIGS. 2D, 6 and 7 illustrate examples of recombination and Table 2 summarizes the findings in multiply infected subjects. Seven of 9 subjects had evidence of recombination within gp160 env (one subject, AD77, could not be analyzed because of excessive virus diversity at a late Fiebig stage). The proportion of recombinants ranged from 0 of 30 sequences in subject 04013211 to 30 of 72 sequences (42%) in subject 701010068. In the latter subject, a longer fragment of the viral genome was amplified so as to include the 3′ half; this allowed us to compare recombination frequencies within gp41 (only), gp160 (only) or the full-length 3′ half genome. The proportion of recombinants in these three regions was 13/72 (18%), 30/72 (42%) and 63.72 (88%), respectively. Recombination breakpoints were more common in sequences flanking gp160 env than within env (FIG. 7C), a finding similar to that reported by Simon-Loriere and colleagues for HIV-1 inter-subtype recombination (Simon-Loriere et al, PLoS Pathog. 5:e1000418 (2009)). In subject 701010068, where 88% of sequences corresponding to only half the viral genome were recombinant, it is likely that nearly all of the full-genome sequences at this time point are recombinant. Since recombination requires an earlier infection event in which a cell is infected by two or more viruses, these findings suggest that in acutely infected humans prior to antibody seroconversion (Fiebig stages III/IV), a substantial fraction of productively infected cells are infected by more than one virus, a circumstance undoubtedly facilitated by initially high virus loads at a time when target cell availability is rapidly declining (Phillips, Science 271:497-499 (1996)).

A final unique aspect to this study was its in-depth analysis of early virus replication kinetics (FIG. 3) and diversification (FIG. 4) in subject AD17 who was exposed to HIV-1 by receptive anal intercourse approximately 6 days before developing symptoms of the acute retroviral syndrome and 14-17 days before peak plasma viremia of 47,600,000 RNA molecules/ml. This exposure to HIV-1 was through a new sexual partner (AD18) who could be proved by phylogenetic analysis to be the source of subject AD17's acute HIV-1 infection (FIG. 1). Assuming a plasma viral load (vL) of 10 RNA copies/ml at the time of symptom onset 6 days after virus infection, then during the peridd between days 6 and 14, vL increased by a factor of ˜10⁶. This implies virus grew exponentially with growth rate r=1.73/day, i.e. exp(1.73*8)˜10⁶. This expansion rate is slower than the expansion rate calculated by Little [50] of 2.0/day but similar to that reported by Stafford (J. Theor. Biol. 203:285-301 (2000)) of 1.67/day. Subject AD17 began HAART on day 17, and between days 17 and 25, vL fell approximately 200-fold. Assuming HAART is nearly 100% effective (Markowitz et al, J. Virol. 77:5037-38 (2003)), then the productively infected cell death rate, δ, can be calculated from the rate of vL decline as In(200)/8=0.66/day. These values can then be used to estimate R₀, the basic reproductive number, as (1+r/δ) exp(rτ), where τ is the intracellular delay phase. If the delay phase is ignored, then R₀=(1+r/δ) and the estimate of R₀ is 3.6. However, if the delay phase is included and it is assumed that τ is one day, then R₀=20.4. This is larger than the estimates in Stafford (J. Theor. Biol. 203:285-301 (2000)). These data support the basic assumptions used in the development of the model of early HIV-1 evolution (Keele et al, Proc. Natl. Acad. Sci. USA 105:7552-57 (2008), Lee et al, J. Theor. Biol. 261:341-60 (2009)), and the genomic integrity and replication competence of the full-length proviral clone pAD 17.1 provides further corroboration of the model.

Only four other transmitted/founder virus molecular clones have been described (Salazar-Gonzalez et al, J. Exp. Med. 206:1273-89 (2009)), and all of these correspond to HIV-1 subtype C viruses resulting from heterosexual transmissions. With the addition of the pAD17.1 clone, molecular proviral clones are available representing male-to-male rectal transmission (pAD17.1), male-to-female vaginal transmission (pZM246F-10; pZM247Fv1; pZM247Fv2), and female-to-male penile transmission (pZM249M-1). All of these viruses are R5 tropic, replicate efficiently in activated human CD4+ T cells but fail to replicate efficiently in monocyte-derived macrophages. Such molecular clones of transmitted/founder viruses should represent a rich resource for studying the biology of HIV-1 transmission and its prevention.

In summary, the findings presented here provide for the first time a comparative, quantitative view of the HIV-1 transmission event in two patient risk groups that dominate the HIV-1 pandemic. In doing so, they highlight both challenges and opportunities confronting candidate vaccines, microbicides, and other prevention modalities. Elucidation of the biological basis of single versus multivariant transmission in MSM and HSX could help advance prevention strategies (Buckheit et al, Antiviral Res. 85:142-58 (2010), Veazey et al, Nat. Med. 9:343-346 (2003), Zhu et al, Nat, Med 15:886-892 (2009)), with quantitative analyses of transmitted/founder viruses representing a potentially valuable new endpoint in vaccine and microbicide trial design and assessment (Rerks-Ngarm et al, N. Engl. J. Med. 361:2209-2220 (2009), Dolin, N. Engl. J. Med. 361:2279-2280 (2009), Boily et al, Lancet Infect. Dis. 8:200-7 (2008), Boily et al, Lancet Infect. Dis. 9:118-29 (2008)).

All documents and other information sources cited above are hereby incorporated in their entirety by reference.

TABLE 1
Demographics, risk group and baseline laboratory data

| | | Geographic | Sexual | Sample | Viral load | CD4 count | | Western | Fiebig |
|---|---|---|---|---|---|---|---|---|---|
| Subject | Subtype | location | partners | date | RNA/ml | cells/μl | ELISA^{a} | blot | stage |
| 04013171 | B | New York | Multiple | Feb. 6, 2002 | 3,700,000 | 213 | R | indet | 4 |
| 04013211 | B | New York | Multiple | Aug. 23, 2002 | 19,900,000 | 846 | R | neg | 3 |
| 04013226 | B | New York | Single | Nov. 20, 2002 | 26,700,000 | 175 | N | neg | 2 |
| 04013240 | B | New York | Multiple | Jan. 21, 2003 | 2,240,000 | 297 | N | neg | 2 |
| 04013242 | B | New York | Multiple | Jan. 23, 2003 | 5,790,000 | 251 | R | indet | 4 |
| 04013291 | B | New York | Multiple | Jun. 4, 2003 | 1,490,000 | 179 | R | pos(p31-) | 5 |
| 04013296 | B | New York | Multiple | Aug. 5, 2003 | 8,050,000 | 395 | N | neg | 2 |
| 04013321 | B | New York | Single | Oct. 10, 2003 | 6,250,000 | 407 | N | neg | 2 |
| 04013327 | B | New York | Single | Jan. 27, 2004 | 8,720,000 | 248 | R | indet | 4 |
| 04013383 | B | New York | Multiple | Apr. 5, 2005 | 584,000 | 531 | N | neg | 2 |
| 04013396 | B | New York | Multiple | Aug. 16, 2005 | 1,600,000 | 581 | R | indet | 4 |
| 04013419 | B | New York | Multiple | Mar. 14, 2006 | 21,200,000 | 226 | N | neg | 2 |
| 04013440 | B | New York | Multiple | Oct. 17, 2006 | >100,000 | 205 | N | neg | 2 |
| 04013446 | B | New York | Single | Nov. 28, 2006 | >100,000 | 438 | R | neg | 3 |
| 04013448 | B | New York | Single | Jan. 19, 2007 | 28,600,000 | 536 | N | neg | 2 |
| AD17 | B | New York | Multiple | Jun. 14, 1999 | 47,600,000 | nos | N | neg | 2 |
| AD75 | B | New York | Multiple | Nov. 6, 2002 | 21,400,000 | nos | N | neg | 2 |
| AD77 | B | New York | Multiple | Nov. 15, 2002 | 130,000 | nos | R | pos(p31-) | 5 |
| AD83 | B | New York | Multiple | Jan. 22, 2003 | 448,000 | nos | R | pos(p31-) | 5 |
| HOBR0961 | B | Alabama | Single | Oct. 31, 1991 | 599,238 | 794 | N | neg | 2 |
| INME0632 | B | Alabama | Single | Aug. 9, 1990 | 2,217,670 | 739 | N | neg | 2 |
| 701010055 | B | North Carolina | nos^{b} | Oct. 5, 2006 | 31,513,812 | 432 | N | neg | 2 |
| 701010068 | B | North Carolina | Multiple | Oct. 24, 2006 | 3,714,386 | 109 | R | indet | 4 |
| 700010106 | B | North Carolina | nos | Oct. 19,2006 | 84,545,454 | 277 | N | neg | 2 |
| 701010027 | B | North Carolina | nos | Aug. 29, 2006 | 194,744 | 542 | R | pos(p31-) | 5 |
| 701010108 | B | North Carolina | nos | Jun. 28, 2007 | 14,711 | 592 | R | pos(p31-) | 5 |
| 700010246 | B | North Carolina | nos | Jun. 7, 2007 | 4,395,721 | 1012 | R | indet | 4 |
| 700010238 | B | North Carolina | nos | May 8, 2007 | 596,908 | 587 | R | pos(p31-) | 5 |

^{a}R—reactive; N—nonreactive.
^{b}nos—not otherwise specified.

TABLE 2
Diversity and model analysis of full length env sequences from 28 acutely infected subjects

| | | | | Maximum | | | Poisson | |
|---|---|---|---|---|---|---|---|---|
| | | No. | Maximum | Hamming | Maximum | APOBEC- | estimated days | |
| | Fiebig | of SGA | nt length | distance | diversity | mediated | since MRCA^{b} | |
| Subject | stage | envs | of env | (HD)^{a} | % | hypermutation | (C.I.) | Lambda^{c} |
| 04013171 | 4 | 23^{g}/86^{h} | 2625 | 179 | 6.82% | No | 662 (525, 792) | 41.370 |
| 04013211 | 3 | 30/30 | 2619 | 9 | 0.34% | No | 54 (44, 64) | 3.380 |
| 04013226 | 2 | 33/33 | 2574 | 5 | 0.19% | Yes | 15 (9, 21) | 0.936 |
| 04013240 | 2 | 33/66 | 2568 | 14 | 0.55% | No | 66 (56,76) | 4.041 |
| 04013242 | 4 | 37/37 | 2580 | 5 | 0.19% | Yes | 16 (10, 22) | 0.992 |
| 04013291 | 5 | 25/25 | 2559 | 8 | 0.31% | No | 61 (53, 70) | 3.737 |
| 04013296 | 2 | 25/25 | 2640 | 7 | 0.27% | No | 34 (26, 42) | 2.113 |
| 04013321 | 2 | 49/49 | 2556 | 8 | 0.31% | Yes | 39 (33, 45) | 2.370 |
| 04013327 | 4 | 24/24 | 2535 | 4 | 0.16% | No | 11 (5, 18) | 0.667 |
| 04013383 | 2 | 23/70 | 2547 | 70 | 2.75% | No | 564 (534, 572) | 33.580 |
| 04013396 | 4 | 39/39 | 2577 | 5 | 0.19% | No | 16 (11, 21) | 0.974 |
| 04013419 | 2 | 27/78 | 2565 | 80 | 3.12% | Yes | 548 (506, 591) | 33.500 |
| 04013440 | 2 | 30/30 | 2580 | 4 | 0.16% | No | 23 (18, 28) | 1.379 |
| 04013446 | 3 | 23/23 | 2594 | 7 | 0.27% | No | 24 (12, 35) | 1.450 |
| 04013448 | 2 | 15/54 | 2625 | 51 | 1.94% | No | 411 (367, 466) | 25.716 |
| AD17 | 2 | 51/51 | 2544 | 4 | 0.16% | Yes | 8 (5, 11) | 0.471 |
| AD75 | 2 | 54/54 | 2556 | 3 | 0.12% | Yes | 9 (6, 13) | 0.555 |
| AD77 | 5 | 40/40 | 2556 | 11 | 0.43% | No | 84 (74, 95) | 5.127 |
| AD83 | 5 | 44/44 | 2568 | 33 | 1.29% | Yes | 66 (36, 95) | 4.000 |
| HOBR0961 | 2 | 42/42 | 2586 | 4 | 0.15% | No | 17 (13, 22) | 1.022 |
| INME0632 | 2 | 46/46 | 2580 | 4 | 0.16% | No | 12 (8, 16) | 0.737 |
| 701010055 | 2 | 28/28 | 2544 | 3 | 0.12% | No | 15 (10, 21) | 0.923 |
| 701010068 | 4 | 17/89 | 2595 | 115 | 4.43% | No | 688 (583, 792) | 42.499 |
| 700010106 | 2 | 40/40 | 2595 | 5 | 0.19% | Yes | 13 (8, 18) | 0.800 |
| 701010027 | 5 | 27/27 | 2571 | 8 | 0.31% | No | 42 (32, 52) | 2.552 |
| 701010108 | 5 | 35/35 | 2553 | 6 | 0.24% | Yes | 39 (34, 45) | 2.376 |
| 700010246 | 4 | 45/45 | 2607 | 6 | 0.23% | Yes | 19 (13, 25) | 1.180 |
| 700010238 | 5 | 38/38 | 2595 | 19 | 0.73% | No | 161 (149, 174) | 9.970 |

| | | | | | No. of | |
|---|---|---|---|---|---|---|
| | Goodness | | | Explanation | transmitted/ | |
| | of fit P | HD fit to | Star | for deviation | founder | |
| Subject | value^{d} | poisson | phylogeny^{e} | from model | viruses | Recombinants^{f} |
| 04013171 | 0.000 | no | no | multiple variant | ≧10 | 20/86 |
| | | | | transmission | | |
| 04013211 | 0.000 | no | no | multiple variant | 2 | 0/30 |
| | | | | transmission | | |
| 04013226 | 0.825 | yes | yes | | 1 | |
| 04013240 | 0.000 | no | no | multiple variant | 3 | 6/66 |
| | | | | transmission | | |
| 04013242 | 0.902 | yes | yes | | 1 | |
| 04013291 | 0.100 | no | no | CTL | 1 | |
| 04013296 | 0.859 | yes | yes | | 1 | |
| 04013321 | 0.212 | yes | no | early stochastic | 1 | |
| | | | | mutations | | |
| 04013327 | 0.390 | yes | yes | | 1 | |
| 04013383 | 0.000 | no | no | multiple variant | 2 | 0/70 |
| | | | | transmission | | |
| 04013396 | 0.897 | yes | yes | | 1 | |
| 04013419 | 0.000 | no | no | multiple variant | 3 | 4/78 |
| | | | | transmission | | |
| 04013440 | 0.116 | yes | yes | | 1 | |
| 04013446 | 0.000 | no | no | early stochastic | 1 | |
| | | | | mutations | | |
| 04013448 | 0.000 | no | no | multiple variant | 4 | 5/54 |
| | | | | transmission | | |
| AD17 | 0.162 | yes | yes | | 1 | |
| AD75 | 0.774 | yes | yes | | 1 | |
| AD77 | 0.151 | no | no | multiple variant | 3 | ndⁱ |
| | | | | transmission | | |
| AD83 | 0.000 | no | no | multiple variant | 3 | 3/44 |
| | | | | transmission | | |
| HOBR0961 | 0.503 | yes | yes | | 1 | |
| INME0632 | 0.845 | yes | yes | | 1 | |
| 701010055 | 0.590 | yes | yes | | 1 | |
| 701010068 | 0.000 | no | no | multiple variant | 7 | 30/72^{j} |
| | | | | transmission | | |
| 700010106 | 0.634 | yes | yes | | 1 | |
| 701010027 | 0.977 | yes | yes | | 1 | |
| 701010108 | 0.099 | no | no | CTL | 1 | |
| 700010246 | 0.574 | yes | yes | | 1 | |
| 700010238 | 0.000 | no | no | multiple variant | 3 | 8/38 |
| | | | | transmission | | |

^{a}HD. Hamming Distance—number of base positions at which two sequences differ.
^{b}MRCA—most recent common ancestor.
^{c}Lamda—mean of the best fitting Poisson found through maximum likelihood method.
^{d}Goodness of fit P value—X² goodness-of-fit test statistic for λ, where p < 0.05 suggests that the observed distribution of mutations is inconsistent with a Poisson.
^{e}Star phylogeny—random virus evolution.
^{f}Recombinants in gp160 env
^{g}Initial sequence set used for statistical comparisons.
^{h}Total number of sequences analyzed.
nd—not done.
ⁱRecombinants in gp160 were 30 out of 72 sequences but in the 3′ half genome were 63 out of 72 sequences.

TABLE 3
Multiplicity of HIV-1 infection in MSM vs heterosexual subjects

| | | | Total | Single variant | | Multiple variant | | | | Number of variants | | |
|---|---|---|---|---|---|---|---|---|---|---|---|---|
| Route of | | Virus | subjects | transmission | | transmission | | p | odds | | | adjusted |
| transmission | Study | subtype | n | n | % | n | % | value | ratio | median | range | median^{a} |
| Heterosexual | Keele [10] | B | 79 | 65 | 82.3% | 14 | 17.7% | | | 1 | 1-4 | 2 |
| | Abrahams [8] | C | 69 | 54 | 78.3% | 15 | 21.7% | | | 1 | 1-5 | 3 |
| | Haaland [9] | A and C | 27 | 22 | 81.5% | 5 | 18.5% | | | 1 | 1-6 | 2 |
| | Total | | 175 | 141 | 80.6% | 34 | 19.4% | | | 1 | 1-6 | 2 |
| MSM | Keele [10] | B | 22 | 13 | 59.1% | 9 | 40.9% | | | 1 | 1-6 | 3 |
| | Li (PLoS Path) | B | 28 | 18 | 64.3% | 10 | 35.7% | | | 1 | 1-10 | 3 |
| | Total | | 50 | 31 | 62.0% | 19 | 38.0% | 0.008 | 2.5 | 1 | 1-10 | 3 |

^{a}Adjusted median values are for multivariant transmissions only.

What is claimed is:

1. An isolated polypeptide comprising an HIV-1 transmitted/founder virus Env sequence, or fragment thereof.

2. The polypeptide according to claim 1 wherein said virus is AD17.1.

3. The polypeptide according to claim 1 wherein said fragment comprises a membrane-proximal external region (MPER) of said Env sequence.

4. The polypeptide according to claim 1 wherein said polypeptide comprises the amino acid sequence set forth in FIG. 9B.

5. An isolated nucleic acid encoding said polypeptide according to claim 1, or said fragment thereof.

6. The nucleic acid according to claim 5 wherein said virus is AD 17.1

7. The nucleic acid according to claim 5 wherein said nucleic acid encodes said amino acid sequence set forth in FIG. 9B.

8. A vector comprising the nucleic acid according to claim 5.

9. The vector according to claim 8 wherein said vector is a viral vector.

10. The vector according to claim 8 wherein said vector is a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, a pox virus vector, a recombinant polio vector, a Salmonella species bacterial vector, a Shigella species bacterial vector, a Venezuelean Equine Encephalitis Virus (VEE) vector, a Semliki Forest Virus vector, or a Tobacco Mosaic Virus vector.

11. A composition comprising the polypeptide according to claim 1 and a carrier.

12. The composition according to claim 11 wherein said composition further comprises an adjuvant.

13. A composition comprising the nucleic acid according to claim 5 and a carrier.

14. The composition according to claim 13 wherein said composition further comprises an adjuvant.

15. A method of inducing an anti-HIV-1 immune response in a mammal comprising administering to said mammal said polypeptide, or said fragment thereof, according to claim 1 in an amount sufficient to effect induction.

16. The method according to claim 15 wherein said virus is AD17.1

17. A method of inducing an anti-HIV-1 immune response in a mammal comprising administering to said mammal said nucleic acid according to claim 5 in an amount and under conditions such that said nucleic acid is expressed, and said polypeptide, or said fragment thereof, is thereby produced, so that said induction is effected.

18. The method according to claim 17 wherein said virus is AD17.1

19. A composition comprising at least one sequence selected from the group consisting of wildtype (WT) HIV-1 transmitted/founder virus gag, env, pol, nef and tat sequences and a carrier.

20. The composition according to claim 19 wherein said virus is AD17.1

21. An isolated antibody specific for a transmitted/founder HIV-1 viral sequence, or antigen binding fragment thereof.

22. The antibody, or fragment thereof, according to claim 21 wherein said virus is AD17.1

23. A composition comprising said antibody, or said fragment thereof, according to claim 21 and a carrier.

24. An isolated molecular clone of a full-length transmitted/founder subtype B HIV-1 virus that is replication competent in human cells, or fragment thereof.

25. The clone according to claim 23 wherein said clone is pAD 17.1.
